(19) United States
(12) Patent Application Publication
(10) Pub. No.: US 2019/0027695 A1
(43) Pub. Date: Jan. 24, 2019

(54) COMPOUND, METHOD FOR PREPARING COMPOUND AND ORGANIC LIGHT EMITTING DISPLAY DEVICE

(71) Applicant: Shanghai Tianma AM-OLED Co., Ltd, Shanghai (CN)

(72) Inventors: Lei ZHANG, Shanghai (CN); Xiangcheng WANG, Shanghai (CN); Wei GAO, Shanghai (CN); Jinghua NIU, Shanghai (CN)

(21) Appl. No.: 16/141,513

(22) Filed: Sep. 25, 2018

(30) Foreign Application Priority Data
Mar. 27, 2018 (CN) 201810258982.2

(51) Int. Cl. H01L 51/00; C07D 519/00; C09K 11/06; C07D 401/14

(52) U.S. Cl. CPC H01L 51/0072; C07D 519/00; C09K 11/06; H01L 51/0067; H01L 51/5016; H01L 51/0061; C07D 401/14; H01L 51/0052; H01L 51/0071

(57) ABSTRACT

The present disclosure relates to the technical field of light emitting materials, in particular to a compound, a method for preparing the compound and an organic light emitting display device. The compound has the following structure:

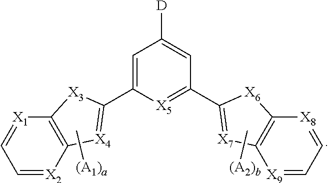

The compound is used in an organic electroluminescent device, can also be used as a host material, a doping material, a hole transport layer material, an electron transport layer material and a cap layer material, can reduce a driving power. This compound can improve a luminous efficiency, brightness, heat stability, color purity and device lifetime.

### CROSS REFERENCE

This application claims the benefit and priority of Chinese Patent Application No. CN201810258982.2, filed on Mar. 27, 2018. The entire disclosure of the above application is incorporated herein by reference.

### FIELD

The present disclosure relates to the technical field of organic electroluminescent materials, in particular to a compound, a method for preparing the compound and an organic light emitting display device.

### BACKGROUND

Mechanisms of light emission in organic light-emitting diodes (OLEDs) include electro fluorescence and electro phosphorescence. Fluorescence occurs when an excited state in a material relaxes to its ground state from an excited singlet state after an excitation light stimulates the material. Phosphorescence is the emission of light when an excited state in a material relaxes to its ground state from an excited triplet state after an excitation light stimulates the material, in this process the excited electron can become trapped in the triplet state with only “forbidden” transitions available to return to the lower energy singlet state. Internal quantum efficiencies in fluorescent materials do not exceed 25%, whereas theoretically internal quantum efficiencies in some electrophosphorescent materials reach 100%.

### SUMMARY

Existing phosphorescent host materials have defects such as low triplet energy levels, small molecular densities, low glass transition temperature and poor heat stability. And in view of these challenges, the present disclosure provides a compound, a method for preparing the compound and an organic light emitting display device.

In one aspect, an embodiment of the present disclosure provides a compound. The compound has a structure illustrated by [chemical formula 1].

[chemical formula 1] is:

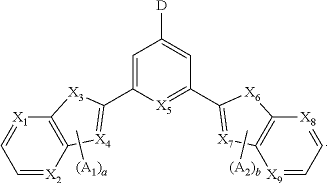

In [chemical formula 1], D represents an electron donor unit. Each of X₁-X₉ is independently selected from a carbon (C) atom or a nitrogen (N) atom. At least one of X₃ and X₄ is an N atom, and at least one of X₆ and X₇ is an N atom. Each of a and b in the subscripts is independently selected from 0 or 1. Each of A₁ and A₂ is independently selected from a group consisting of a substituted or an unsubstituted C₁-C₂₀ alkyl, a substituted or an unsubstituted C₃-C₂₀ cycloalkyl, a substituted or an unsubstituted C₁-C₂₀ alkoxy, a substituted or an unsubstituted C₃-C₂₀ heterocyclic, a substituted or an unsubstituted C₆-C₄₀ aryl, a substituted or an unsubstituted C₁₀-C₄₀ fused aromatic group, and a substituted or an unsubstituted C₄-C₄₀ heteroaryl.

According to one implementation of the present disclosure, X₃ and X₆ are same groups, X₄ and X₇ are same groups, X₁ and X₈ are same groups, X₂ and X₉ are same groups, and A₁ and A₂ may be same or different substituents.

According to one implementation of the present disclosure, the compound is selected from one of the following:

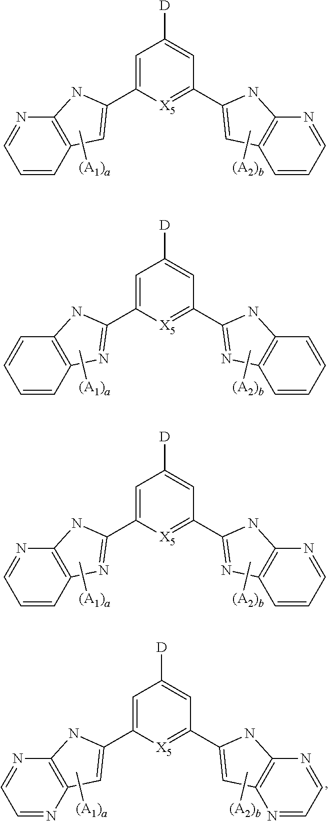

where D represents an electron donor unit, X₅ is selected from a C atom or an N atom, each of a and b in the subscripts is independently selected from 0 or 1, and each of A₁ and A₂ is independently selected from a group consisting of a substituted or an unsubstituted C₁-C₂₀ alkyl, a substituted or an unsubstituted C₃-C₂₀ cycloalkyl, a substituted or an unsubstituted C₁-C₂₀ alkoxy, a substituted or an unsubstituted C₃-C₂₀ heterocyclic, a substituted or an unsubstituted C₆-C₄₀ aryl, a substituted or an unsubstituted C₁₀-C₄₀ fused aromatic group, and a substituted or an unsubstituted C₄-C₄₀ heteroaryl.

According to one implementation of the present disclosure, the unit D is selected from a group consisting of an anilino, an aniline derivative substituent, a carbazolyl, a carbazole derivative substituent, an acridinyl and an acridine derivative substituent.

According to one implementation of the present disclosure, the unit D is selected from one of following: anilino or an aniline derivative substituent:

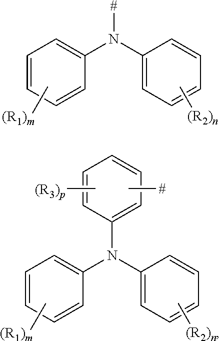

where each of m, n and p in the subscripts is independently selected from 0, 1, 2 or 3; the symbol # represents a location of connection with the heteroaryl in [chemical formula 1]; and each of R₁, R₂ and R₃ is independently selected from a group consisting of a hydrogen atom, a substituted or an unsubstituted C₁-C₃₀ alkyl, a substituted or an unsubstituted silylene, a substituted or an unsubstituted C₁-C₃₀ alkoxy, a substituted or an unsubstituted C₆-C₃₀ aryl, and, a substituted or an unsubstituted C₁₀-C₃₀ fused aromatic group.

According to one implementation of the present disclosure, the unit D is selected from one of following: carbazolyls or carbazole derivative substituents:

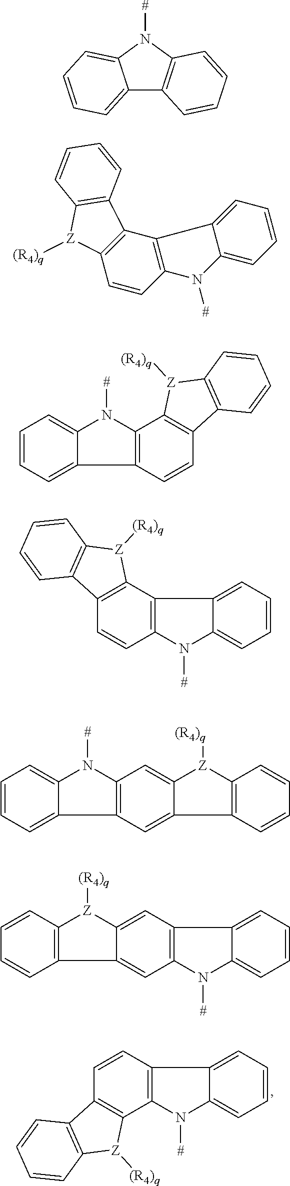

where Z is selected from a C atom, an N atom, an oxygen (O) atom, a sulfur (S) atom or a silicon (Si) atom; each of q in the subscripts is independently selected from 0, 1, 2 or 3; the symbol # represents a location linked with the heteroaryl in [chemical formula 1]; and R₄ is independently selected from a group consisting of a hydrogen atom, a substituted or an unsubstituted C₁-C₃₀ alkyl, a substituted or an unsubstituted silylene, a substituted or an unsubstituted C₁-C₃₀ alkoxy, a substituted or an unsubstituted C₆-C₃₀ aryl and a substituted or an unsubstituted C₁₀-C₃₀ fused aromatic group.

According to one implementation of the present disclosure, the unit D is selected from one of following: acridinyls or acridine derivative substituents:

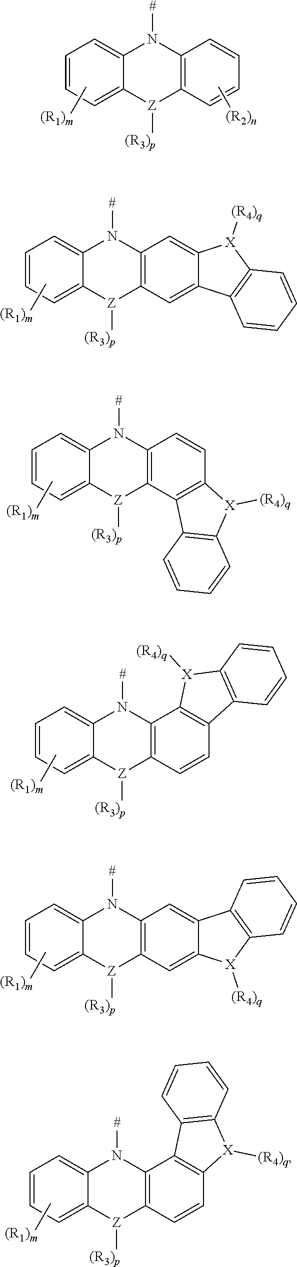

where Z is selected from a C atom, an N atom, an O atom, an S atom or a Si atom; X is selected from a group consisting of a C atom, an N atom, an O atom and an S atom; each of m, n, p and q in the subscripts is independently selected from 0, 1, 2 or 3; the symbol # represents a location linked with the heteroaryl in [chemical formula 1]; and each of R₁, R₂, R₃ and R₄ is independently selected from a group consisting of a hydrogen atom, a substituted or an unsubstituted C₁-C₃₀ alkyl, a substituted or an unsubstituted silylene, a substituted or an unsubstituted C₁-C₃₀ alkoxy, a substituted or an unsubstituted C₆-C₃₀ aryl and a substituted or an unsubstituted C₁₀-C₃₀ fused aromatic group.

According to one implementation of the present disclosure, the unit D is selected from one of following:

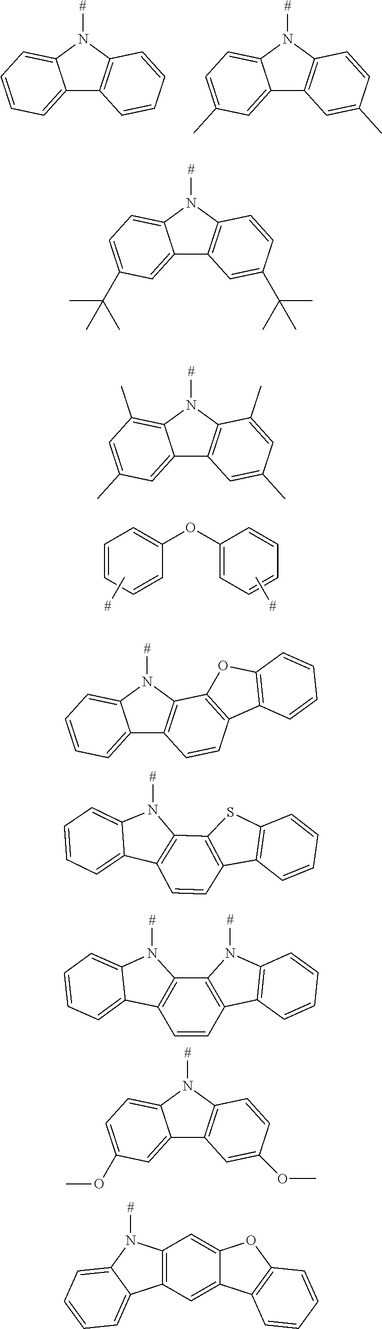

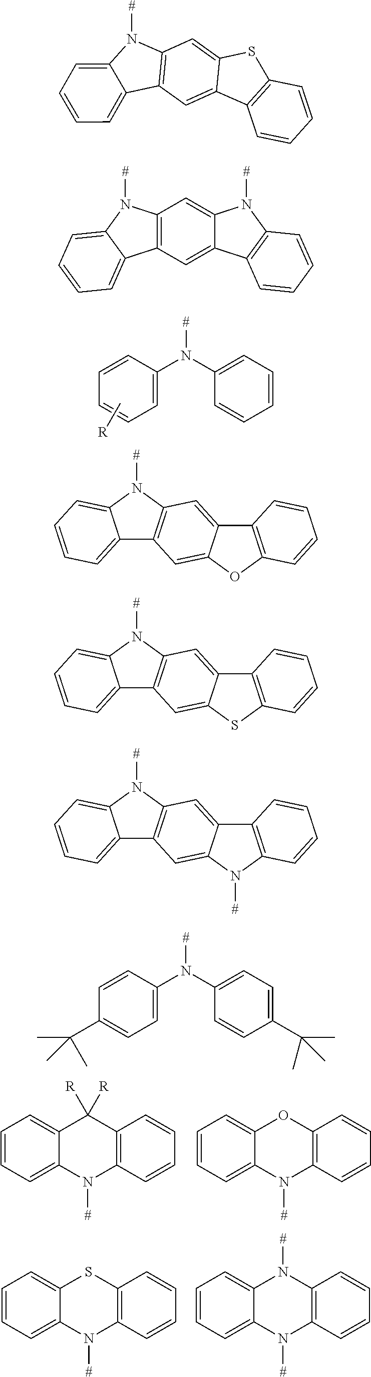

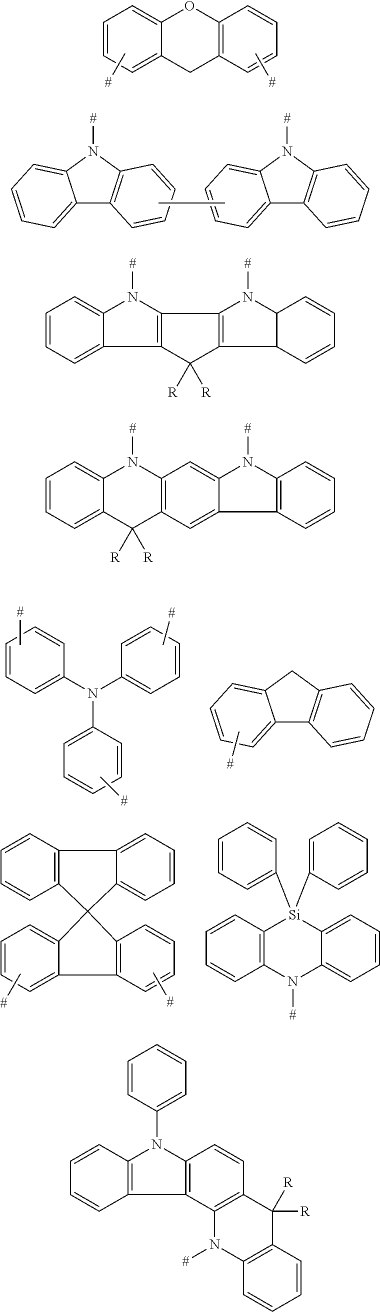

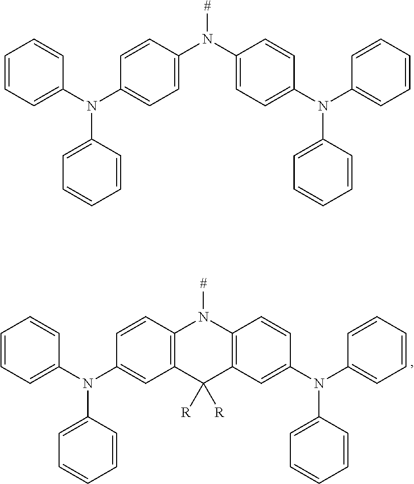

where R in each structural formula is independently selected from a group consisting of an hydrogen atom, a substituted or an unsubstituted C₁-C₂₀ alkyl, a substituted or an unsubstituted C₃-C₂₀ cycloalkyl, a substituted or an unsubstituted C₁-C₂₀ alkoxy, a substituted or an unsubstituted C₃-C₂₀ heterocyclic, a substituted or an unsubstituted C₆-C₄₀ aryl and a substituted or an unsubstituted C₅-C₄₀ heteroaryl. The symbol # represents a location where unit D is able to connect with the heteroaryl in [chemical formula 1].

According to one implementation of the present disclosure, the compound is selected from one of the following chemical compounds:

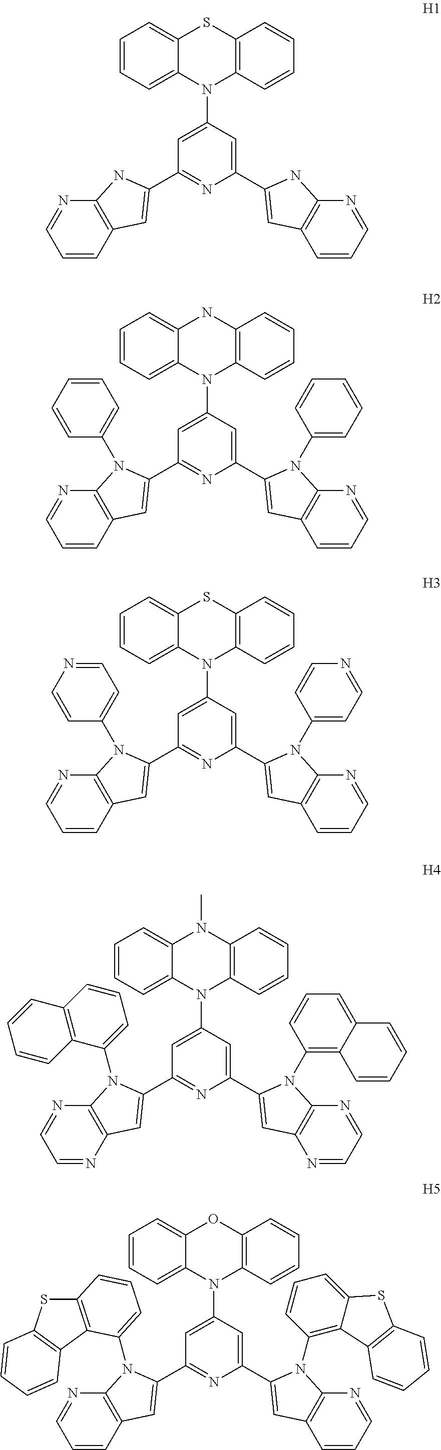 [SMILES: c1ccc2c(c1)Sc1ccccc1N2c1cc(-c2cc3cccnc3[nH]2)nc(-c2cc3cccnc3[nH]2)c1 ; c1ccc(-n2c(-c3cc(N4c5ccccc5Nc5ccccc54)cc(-c4cc5cccnc5n4-c4ccccc4)n3)cc3cccnc32)cc1 ; c1ccc2c(c1)Sc1ccccc1N2c1cc(-c2cc3cccnc3n2-c2ccncc2)nc(-c2cc3cccnc3n2-c2ccncc2)c1 ; CN1c2ccccc2N(c2cc(-c3cc4nccnc4n3-c3cccc4ccccc34)nc(-c3cc4nccnc4n3-c3cccc4ccccc34)c2)c2ccccc21 ; c1ccc2c(c1)Oc1ccccc1N2c1cc(-c2cc3cccnc3n2-c2cccc3sc4ccccc4c23)nc(-c2cc3cccnc3n2-c2cccc3sc4ccccc4c23)c1]

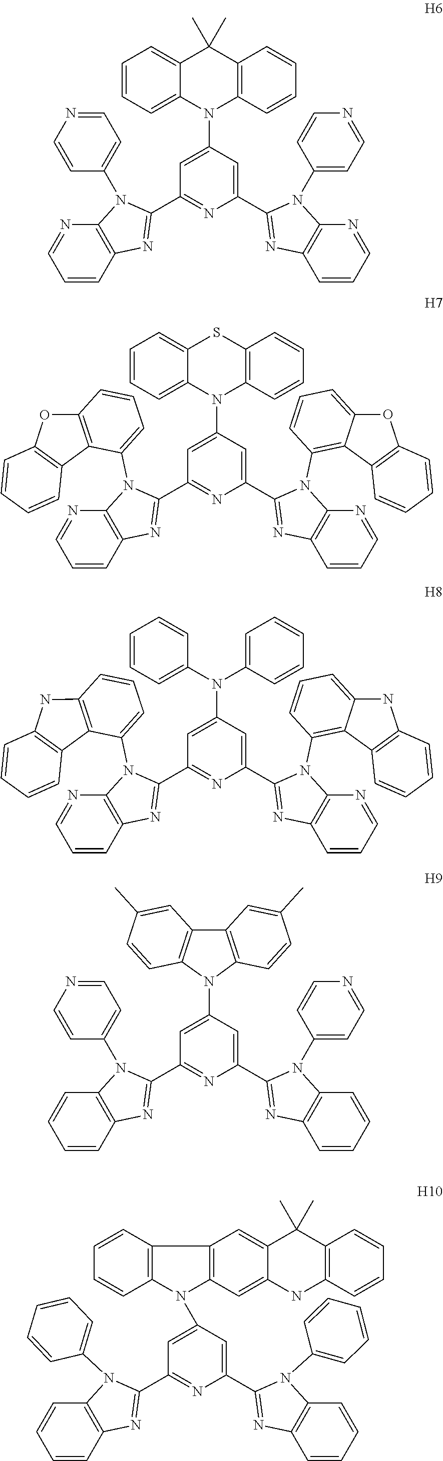

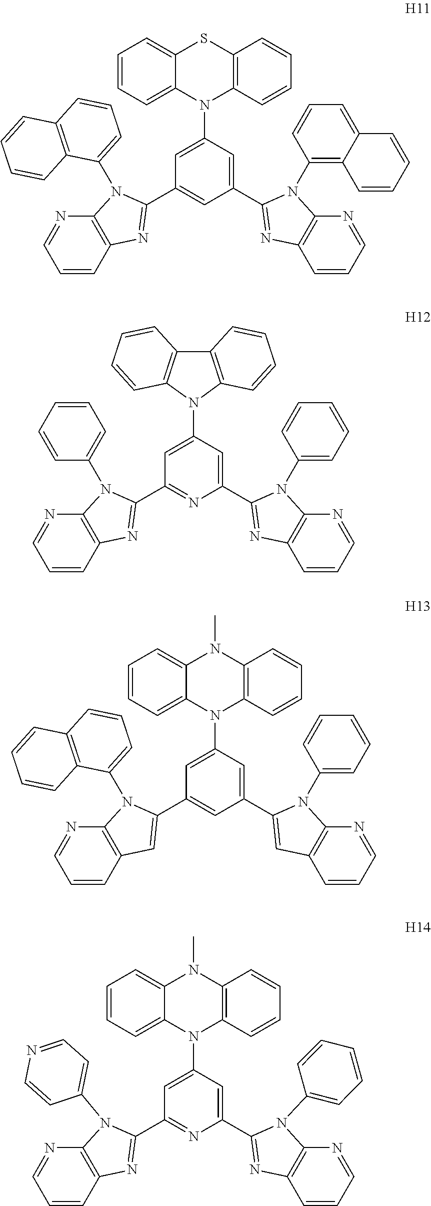 [SMILES: c1ccc2c(c1)Sc1ccccc1N2c1cc(-c2nc3cccnc3n2-c2cccc3ccccc23)cc(-c2nc3cccnc3n2-c2cccc3ccccc23)c1 ; c1ccc(-n2c(-c3cc(-n4c5ccccc5c5ccccc54)cc(-c4nc5cccnc5n4-c4ccccc4)n3)nc3cccnc32)cc1 ; CN1c2ccccc2N(c2cc(-c3cc4cccnc4n3-c3ccccc3)cc(-c3cc4cccnc4n3-c3cccc4ccccc34)c2)c2ccccc21 ; CN1c2ccccc2N(c2cc(-c3nc4cccnc4n3-c3ccccc3)nc(-c3nc4cccnc4n3-c3ccncc3)c2)c2ccccc21]

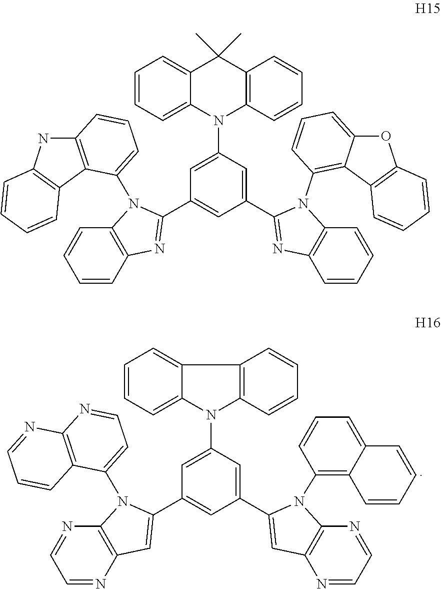 [SMILES: CC1(C)c2ccccc2N(c2cc(-c3nc4ccccc4n3-c3cccc4[nH]c5ccccc5c34)cc(-c3nc4ccccc4n3-c3cccc4oc5ccccc5c34)c2)c2ccccc21 ; c1ccc2c(-n3c(-c4cc(-c5cc6nccnc6n5-c5ccnc6ncccc56)cc(-n5c6ccccc6c6ccccc65)c4)cc4nccnc43)cccc2c1]

In another aspect, the embodiment of the present disclosure further provides a method for preparing the compound. The method includes the following steps expressed with [chemical reaction formula 1] to [chemical reaction formula 4].

Step 1: activing a

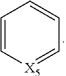

Step 2: reacting with an activated

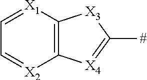

and an activated

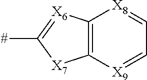

to obtain an intermediate I; where the intermediate I reacts with an activated group D to obtain an intermediate II; and where the intermediate II reacts with an activated A₁ and an activated A₂ to obtain the compound.

[chemical reaction formula 1] is:

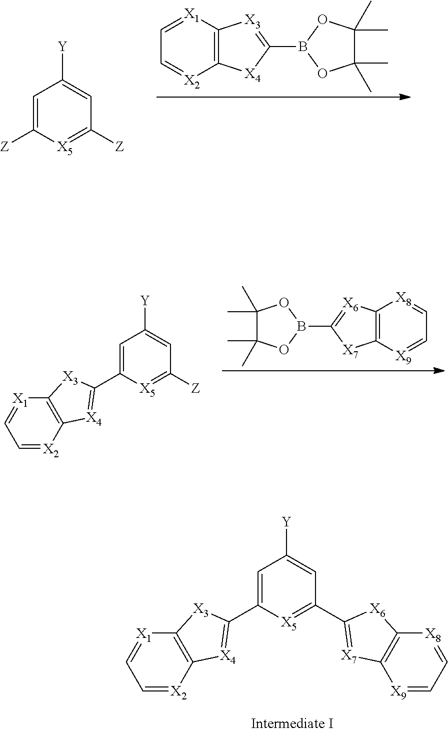

[chemical reaction formula 2] is:

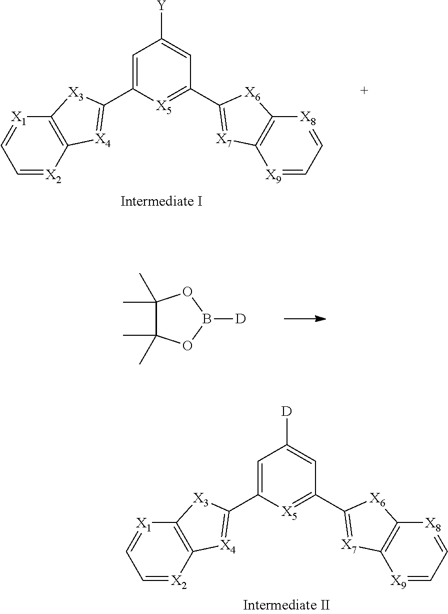

[chemical reaction formula 3] is:

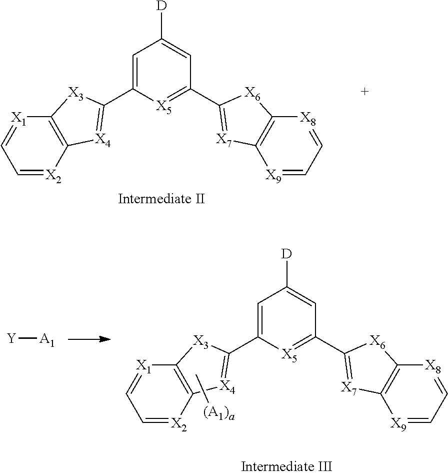

[chemical reaction formula 4] is:

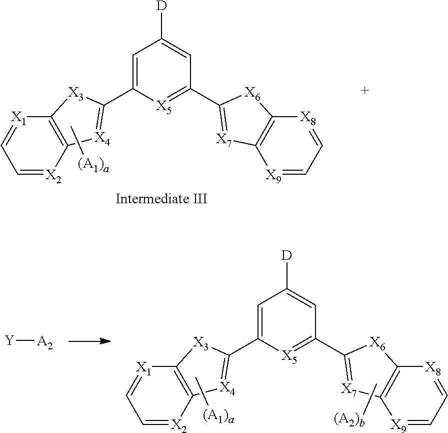

In the above-mentioned [chemical reaction formula 1] to [chemical reaction formula 4], Y and Z represent two different halogens, respectively, Y is selected from iodine (I) or bromine (Br), and Z is selected from Br or chlorine (Cl).

In another aspect, the embodiment of the disclosure of the present disclosure further provides an organic light emitting display device. The organic light emitting display device includes an organic electroluminescent device. The organic electroluminescent device includes an organic function layer. The organic function layer includes one or more organic film layers, at least one of the organic film layers is a light emitting layer, and the organic function layer contains the above-mentioned compound.

For example, the display device according to the embodiment of the present disclosure may be a mobile phone display screen, a computer display screen, or a liquid crystal television display screen.

According to one implementation of the embodiment, a phosphorescent host material of the light emitting layer contains the compound.

According to one implementation of the embodiment, a doping material of the light emitting layer contains the compound.

According to one implementation of the embodiment, the organic function layer further includes an electron transport layer and a material of the electron transport layer contains the compound.

According to one implementation of the embodiment, the organic function layer further includes a hole transport layer and a material of the hole transport layer contains the compound.

According to one implementation of the embodiment, the organic function layer further includes a cap layer (CPL) and a material of the cap layer contains the compound.

According to one implementation of the embodiment, the organic electroluminescent device further includes a first electrode and a second electrode over the organic function layer. The organic function layer is over the first electrode.

### BRIEF DESCRIPTION OF THE DRAWINGS

FIG. 1 illustrates HOMO and LUMO energy level distribution diagrams of a compound H3, where (A) in FIG. 1 is an HOMO energy level distribution diagram of the compound H3 and (B) in FIG. 1 is an LUMO energy level distribution diagram of the compound H3.

FIG. 2 illustrates a structural schematic diagram of an organic electroluminescent device according to the embodiment of the present disclosure.

FIG. 3 illustrates a schematic diagram of a display screen of a mobile phone.

In the drawings, the reference sign 1 refers to a substrate, the reference sign 2 refers to a first electrode, the reference sign 3 refers to an organic function layer, the reference sign 4 refers to a second electrode, and the reference sign 5 refers to a display screen.

### DETAILED DESCRIPTION OF AN EMBODIMENT

Implementations described below are just used for explaining the embodiment of the present disclosure instead of restricting the content of the present disclosure. The embodiment of present disclosure will be further described below in combination with the implementations.

The embodiment of the present disclosure provides a compound, a method for preparing the compound and an organic light emitting display device.

In one aspect, the embodiment of the present disclosure provides a compound. The structure of the compound is illustrated by [chemical formula 1].

[chemical formula 1] is

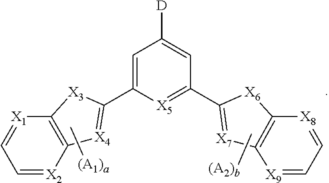

In [chemical formula 1], D represents an electron donor unit. Each of X₁-X₉ is independently selected from a C atom or an N atom, at least one of X₃ and X₄ is an N atom, and at least one of X₆ and X₇ is an N atom. Each of a and b in the subscripts is independently selected from 0 or 1. Each of A₁ and A₂ is independently selected from a group consisting of a substituted or an unsubstituted C₁-C₂₀ alkyl, a substituted or an unsubstituted C₃-C₂₀ cycloalkyl, a substituted or an unsubstituted C₁-C₂₀ alkoxy, a substituted or an unsubstituted C₃-C₂₀ heterocyclic, a substituted or an unsubstituted C₆-C₄₀ aryl, a substituted or an unsubstituted C₁₀-C₄₀ fused aromatic group, and a substituted or an unsubstituted C₄-C₄₀ heteroaryl.

The C₁-C₂₀ alkyl may be selected from saturated aliphatic hydrocarbyls such as a methyl, an ethyl, an n-propyl, an isopropyl, an n-butyl, a sec-butyl and a tent-butyl. The C₁-C₂₀ alkyl may have a substituent or not have a substituent.

The C₃-C₂₀ cycloalkyl may be selected from saturated cycloalkyls such as a cyclopropyl, a cyclopentyl, a cyclohexyl, a cycloheptyl, a cyclooctyl, a norbornyl and an adamantyl. The C₃-C₂₀ cycloalkyl may have a substituent or not have a substituent.

The C₁-C₂₀ alkoxy may be selected from aliphatic alkoxys such as a methoxy, an ethoxy and a propoxy. The C₁-C₂₀ alkoxy may have a substituent or not have a substituent.

The C₃-C₂₀ heterocyclic may be selected from functional groups each having a heteroatom different from a carbon atom in a ring, such as a pyranoid ring, a piperidine ring and a cyclic amide. The C₃-C₂₀ heterocyclic may have a substituent or not have a substituent.

The C₆-C₄₀ aryl may be selected from aromatic hydrocarbyls such as a phenyl, a biphenyl, a 9,9-fluorenyl and a benzene terphenyl. The C₆-C₄₀ aryl may have a substituent or not have a substituent.

The C₁₀-C₄₀ fused aromatic group may be selected from fused cycloaryls such as a naphthyl, an anthryl, a phenanthryl, a 9,10-benzophenanthryl, a 1,2-benzophenanthryl, an acenaphthyl, a perylene, a pyrenyl and an indenyl. The C₁₀-C₄₀ fused aromatic group may have a substituent or not have a substituent.

The C₅-C₄₀ heteroaryl may be selected from a furyl, a thienyl, a pyrryl, an oxazolyl, a thiazolyl, a pyrazolyl, a pyranyl, an imidazolyl, a pyridyl, a pyrazinyl, a pyrimidyl, a 1,3,4-oxadiazolyl, a 1,2,4-triazolyl, a 1,3,5-triazinyl, an indolyl, a benzimidazolyl, a dibenzofuryl, a dibenzothienyl, a carbazolyl, a quinolyl, a quinoxalinyl, a phenanthrolyl, a phenazinyl and a pyridazinyl. The C₅-C₄₀ heteroaryl may have a substituent or not have a substituent.

When the abovementioned C₁-C₂₀ alkyl, the C₃-C₂₀ cycloalkyl, the C₁-C₂₀ alkoxy, the C₃-C₂₀ heterocyclic, the C₆-C₄₀ aryl, the C₁₀-C₄₀ fused aromatic group and the C₅-C₄₀ heteroaryl each have a substituent, the substituent may be selected from an alkyl, a cycloalkyl, an alkoxy, a heterocyclic, an aryl, a fused aromatic group, a heteroaryl, and etc.

For the C₁-C₂₀ alkyl, the C₃-C₂₀ cycloalkyl, the C₁-C₂₀ alkoxy, the C₃-C₂₀ heterocyclic, the C₆-C₄₀ aryl, the C₁₀-C₄₀ fused aromatic group and the C₅-C₄₀ heteroaryl which are mentioned elsewhere herein shall also be understood as the same as above.

According to one implementation of the present disclosure, X₃ and X₆ are same groups, X₄ and X₇ are same groups, X₁ and X₈ are same groups, X₂ and X₉ are same groups, and A₁ and A₂ may be same or different substituents.

According to one implementation of the present disclosure, the compound is selected from one of the following:

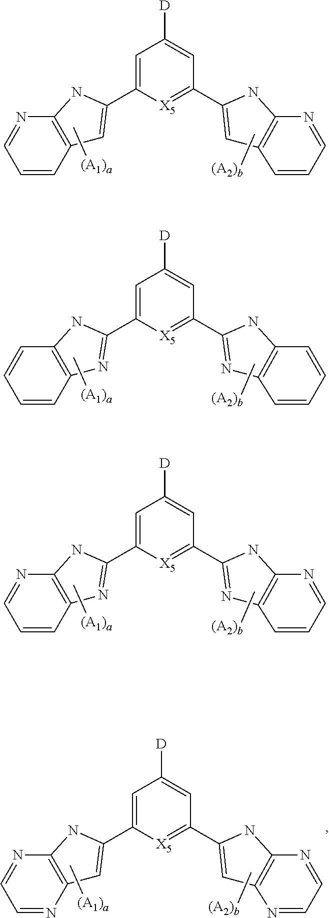

where D represents an electron donor unit, X₅ is selected from a C atom and N atom, each of a and b in the subscripts is independently selected from 0 or 1, and each of A₁ and A₂ is independently selected from a group consisting of a substituted or an unsubstituted C₁-C₂₀ alkyl, a substituted or an unsubstituted C₃-C₂₀ cycloalkyl, a substituted or an unsubstituted C₁-C₂₀ alkoxy, a substituted or an unsubstituted C₃-C₂₀ heterocyclic, a substituted or an unsubstituted C₆-C₄₀ aryl, a substituted or an unsubstituted C₁₀-C₄₀ fused aromatic group, and a substituted or an unsubstituted C₄-C₄₀ heteroaryl.

According to one implementation of the present disclosure, the unit D is selected from a group consisting of an anilino, an aniline derivative substituent, a carbazolyl, a carbazole derivative substituent, an acridinyl and an acridine derivative substituent.

According to one implementation of the present disclosure, the unit D is selected from one of following: anilino or an aniline derivative substituent:

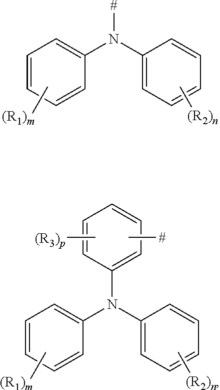

where each of m, n and p in the subscripts is independently selected from 0, 1, 2 or 3; the symbol # represents a location of connection with the heteroaryl in [chemical formula 1]; and each of R₁, R₂ and R₃ is independently selected from a group consisting of a hydrogen atom, a substituted or an unsubstituted C₁-C₃₀ alkyl, a substituted or an unsubstituted silylene, a substituted or an unsubstituted C₁-C₃₀ alkoxy, a substituted or an unsubstituted C₆-C₃₀ aryl, and, a substituted or an unsubstituted C₁₀-C₃₀ fused aromatic group.

According to one implementation of the present disclosure, the unit D is selected from one of following: carbazolyl or carbazole derivative substituents:

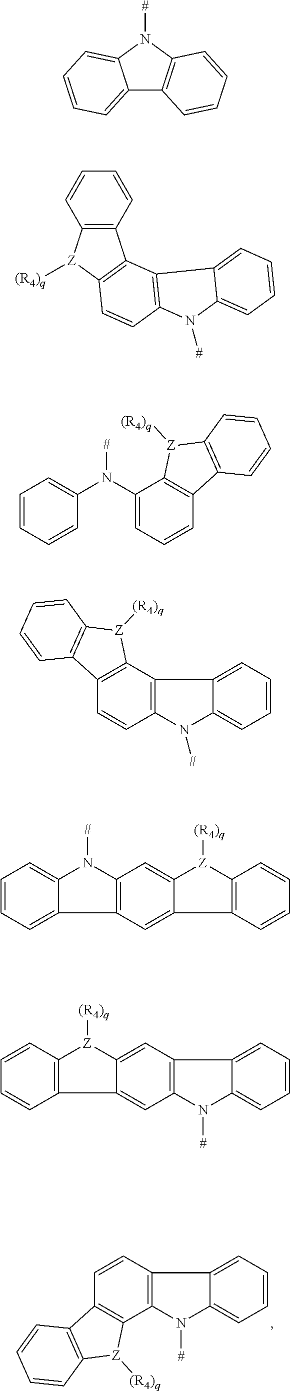

where Z is selected from a C atom, an N atom, an O atom, an S atom or a Si atom; each of q in the subscripts is independently selected from 0, 1, 2 or 3; the symbol # represents a location linked with the heteroaryl in [chemical formula 1]; and R₄ is independently selected from a group consisting of a hydrogen atom, a substituted or an unsubstituted C₁-C₃₀ alkyl, a substituted or an unsubstituted silylene, a substituted or an unsubstituted C₁-C₃₀ alkoxy, a substituted or an unsubstituted C₆-C₃₀ aryl and a substituted or an unsubstituted C₁₀-C₃₀ fused aromatic group.

According to one implementation of the present disclosure, the unit D is selected from one of following: acridinyl or acridine derivative substituents:

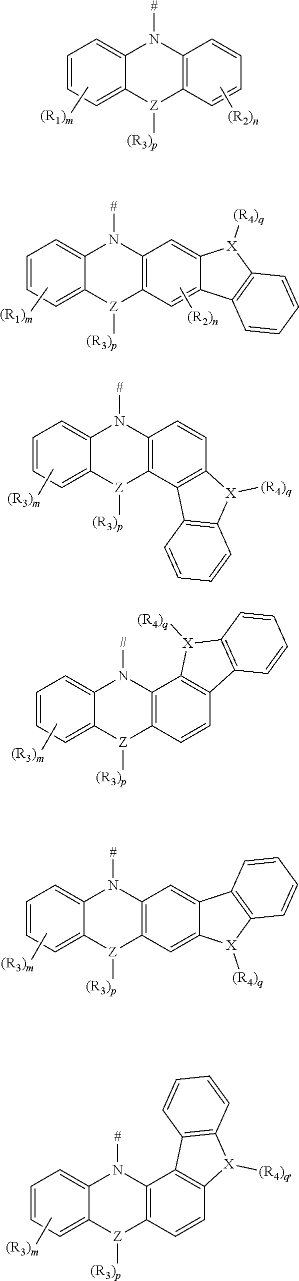

where Z is selected from a C atom, an N atom, an O atom, an S atom or a Si atom; X is selected from a C atom, an N atom, an O atom or an S atom; each of m, n, p and q in the subscripts is independently selected from 0, 1, 2 or 3; the symbol # represents a location linked with the heteroaryl in [chemical formula 1]; and each of R₁, R₂, R₃ and R₄ is independently selected from a group consisting of a hydrogen atom, a substituted or an unsubstituted C₁-C₃₀ alkyl, a substituted or an unsubstituted silylene, a substituted or an unsubstituted C₁-C₃₀ alkoxy, a substituted or an unsubstituted C₆-C₃₀ aryl and a substituted or an unsubstituted C₁₀-C₃₀ fused aromatic group.

According to one implementation of the present disclosure, the unit D is selected from one of the following:

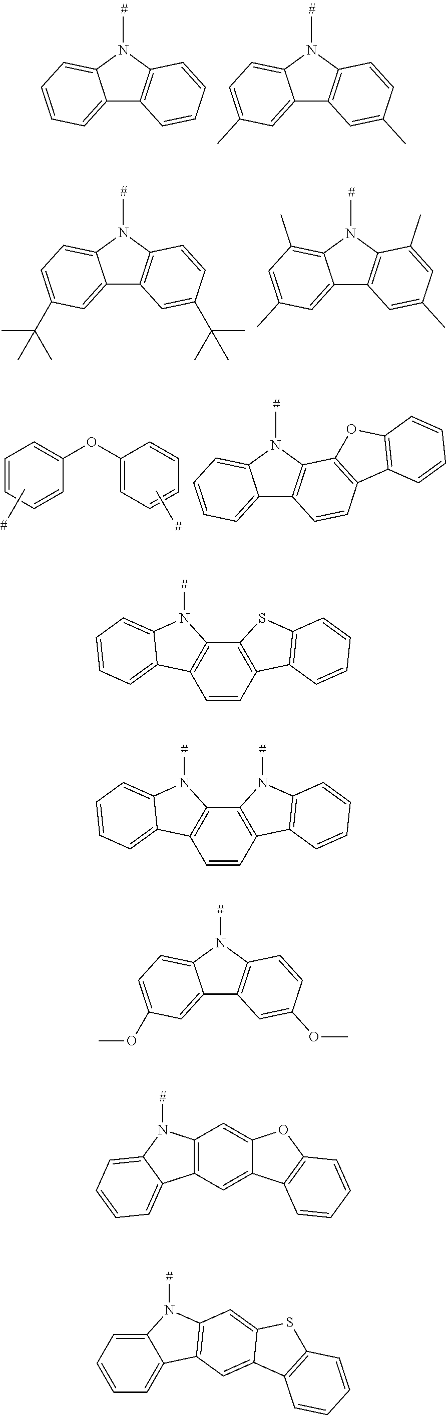

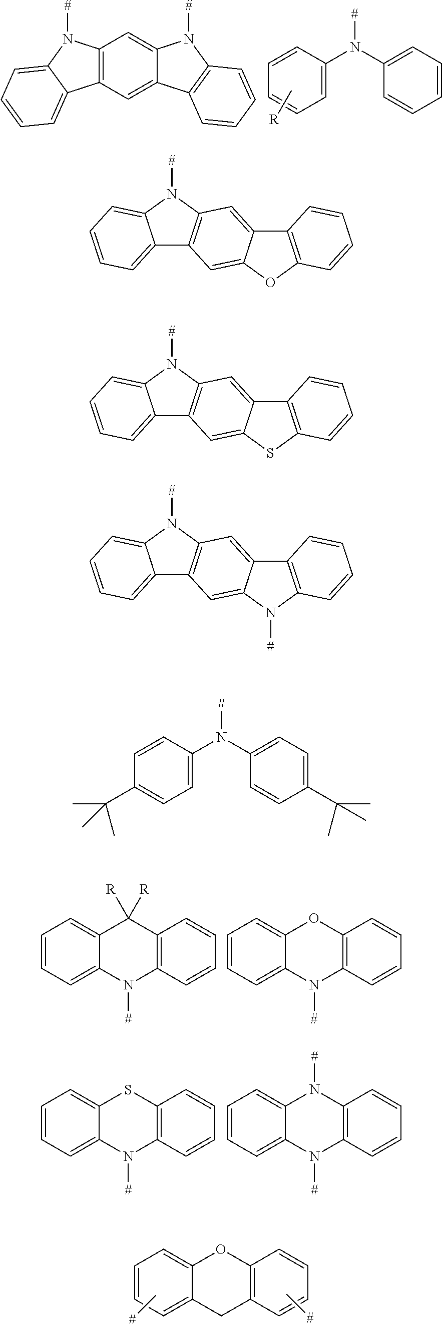

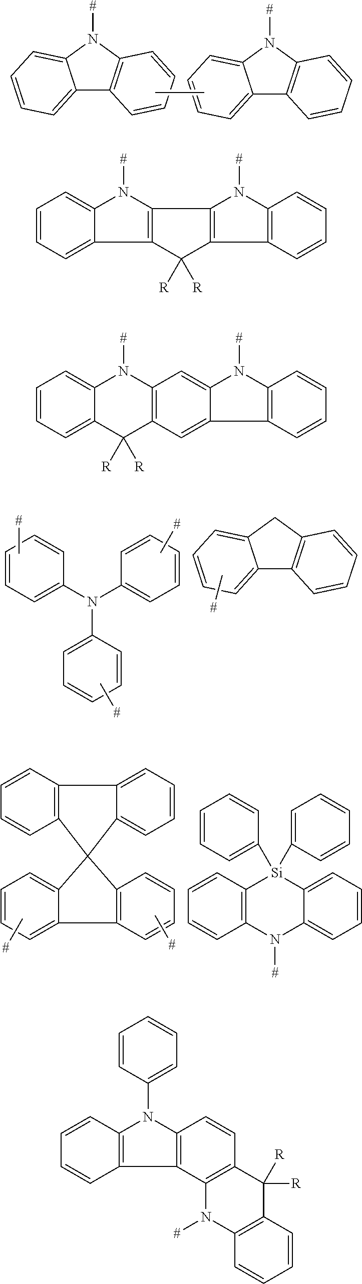

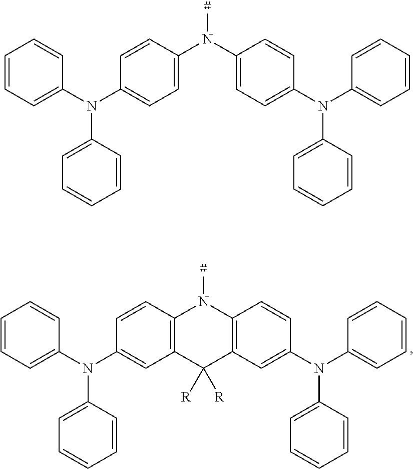

where R in each structural formula is independently selected from a group consisting of an hydrogen atom, a substituted or an unsubstituted C₁-C₂₀ alkyl, a substituted or an unsubstituted C₃-C₂₀ cycloalkyl, a substituted or an unsubstituted C₁-C₂₀ alkoxy, a substituted or an unsubstituted C₃-C₂₀ heterocyclic, a substituted or an unsubstituted C₆-C₄₀ aryl and a substituted or an unsubstituted C₅-C₄₀ heteroaryl. The symbol # represents a location where unit D is able to connect with the heteroaryl in [chemical formula 1].

Herein “the symbol # represents a location where unit D is able to connect with the heteroaryl in [chemical formula 1]” refers to that, when two or more # exist in one chemical structure, any one of these # may be used as a position where the unit D is linked with the heteroaryl in [chemical formula 1].

According to one implementation of the present disclosure, the compound is selected from one of H1 to H16.

In another aspect, the embodiment of the present disclosure provides a method for preparing the compound. The method includes the following steps expressed with [chemical reaction formula 1] to [chemical reaction formula 4].

Step 1: activating a

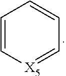

Step 2: reacting with an activated

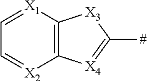

and an activated

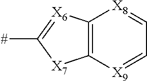

to obtain an intermediate I; where the intermediate I reacts with an activated group D to obtain an intermediate II; and where the intermediate II reacts with an activated A₁ and an activated A₂ to obtain the compound.

[chemical reaction formula 1] is:

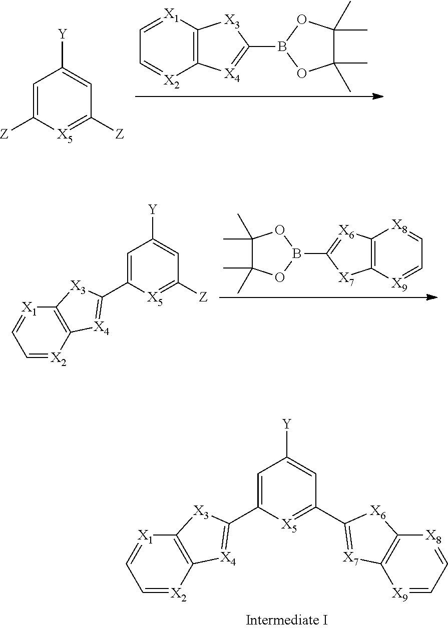

[chemical reaction formula 2] is:

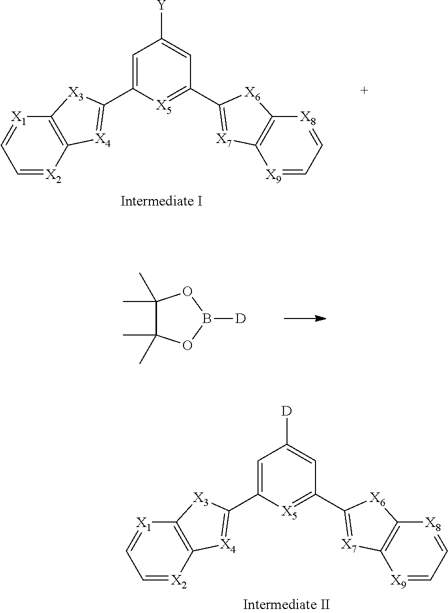

[chemical reaction formula 3] is:

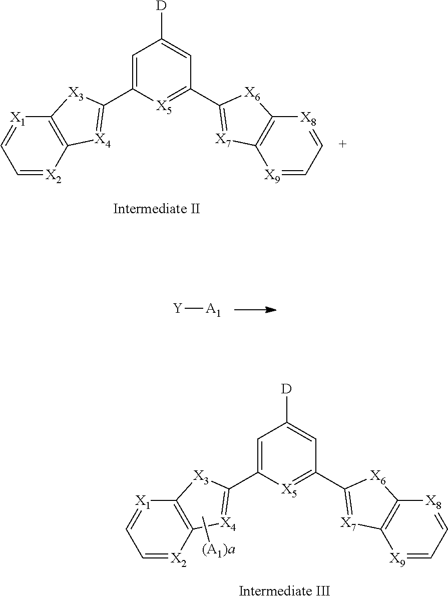

[chemical reaction formula 4] is:

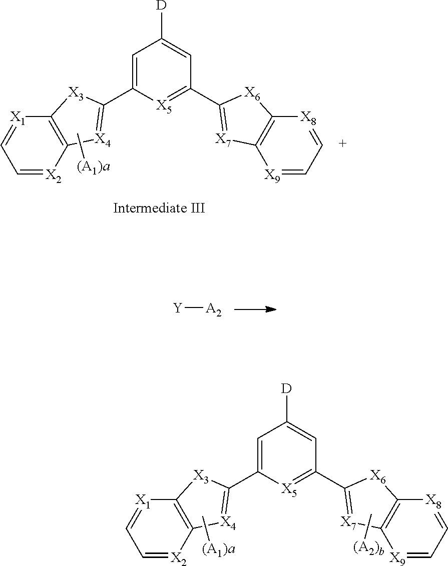

In the above-mentioned [chemical reaction formula 1] to [chemical reaction formula 4], Y and Z represent two different halogens, respectively, Y is selected from I or Br, and Z is selected from Br or Cl.

When X₃ and X₆ are same groups, X₄ and X₇ are same groups, X₁ and X₈ are same groups, X₂ and X₉ are same groups, and A₁ and A₂ may be same or different substituents.

When A₁ and A₂ are same groups, i.e., substituents on left and right sides of

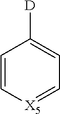

are the same, the intermediate II reacts with Y-A₁ to obtain the compound.

When A₁ and A₂ are different substituents, the intermediate II reacts with Y-A₁ to obtain an intermediate III, and thereafter the intermediate III reacts with Y-A₂ to obtain the compound.

Methods for preparing a plurality of specific compounds and their results are provided below.

Structures of intermediates A₁-A₄ involved in the preparation processes are as shown by Table 1. For methods of preparing the intermediates A₁-A₄, please refer to [chemical reaction formula 1].

TABLE 1
Structures of the intermediates A₁-A₄

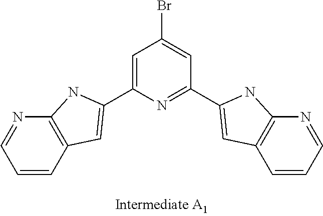 [SMILES: Brc1cc(-c2cc3cccnc3[nH]2)nc(-c2cc3cccnc3[nH]2)c1]

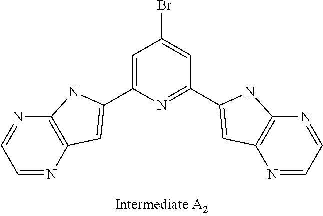 [SMILES: Brc1cc(-c2cc3nccnc3[nH]2)nc(-c2cc3nccnc3[nH]2)c1]

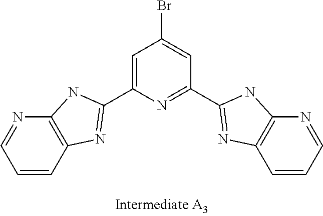 [SMILES: Brc1cc(-c2nc3cccnc3[nH]2)nc(-c2nc3cccnc3[nH]2)c1]

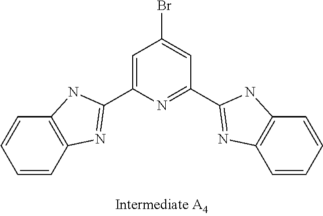 [SMILES: Brc1cc(-c2nc3ccccc3[nH]2)nc(-c2nc3ccccc3[nH]2)c1]

Synthesis of a compound H3

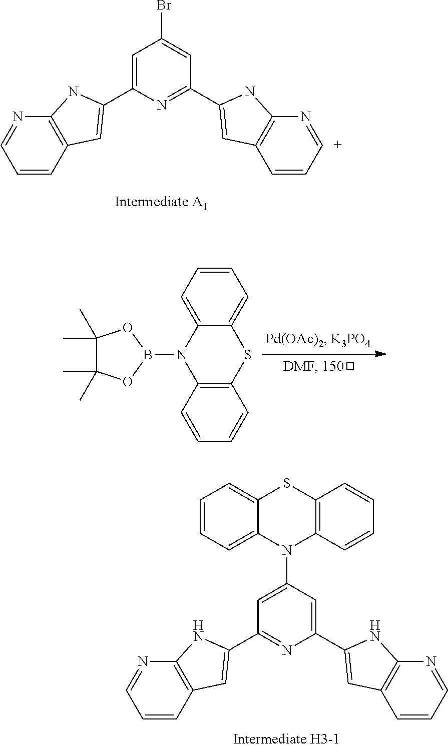

Step 1: nitrogen is fed into a 250 ml three-necked flask, an intermediate A₁ (0.03 mol), phenothiazine borate (0.036 mmol) and palladium acetate (0.0003 mol) are sequentially added into 150 ml of DMF, stirring for mixing is performed during the adding, then aqueous solution of K₃PO₄ (0.045 mol) is added, reflux reaction is performed for 10 h at a temperature of 130° C., natural cooling to room temperature is performed, 100 ml of water is added, an obtained mixture is filtered and dried in a vacuum drying oven, and obtained residues are enabled to pass through a silica gel column for further separation and purification to obtain an intermediate H3-1.

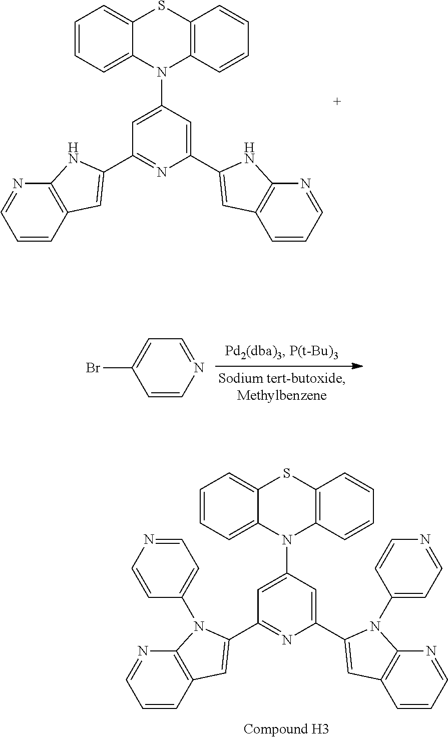 [SMILES: c1ccc2c(c1)Sc1ccccc1N2c1cc(-c2cc3cccnc3[nH]2)nc(-c2cc3cccnc3[nH]2)c1 ; Brc1ccncc1 ; c1ccc2c(c1)Sc1ccccc1N2c1cc(-c2cc3cccnc3n2-c2ccncc2)nc(-c2cc3cccnc3n2-c2ccncc2)c1]

Step 2: the intermediate H3-1 (0.03 mol), 4-bromo-pyridine (0.042 mol), Pd₂(dba)₃ (0.00018 mol), sodium tert-butoxide (0.00018 mol) and tri-tert-butylphosphine (0.00018 mol) are added into 100 ml of methylbenzene under the protection of nitrogen atmosphere, the mixture is stirred, and the mixed solution of the reactants is heated for refluxing for 10-15 h; sampling and plate spotting are performed till complete reaction; natural cooling and filtration are performed, rotary evaporation is performed to filtrate and the residue is enabled to pass through a silica gel column to obtain a compound H3.

Elementary analysis result of compound H3 (molecular formula: C₄₁H₂₆N₈S): theoretical value: C, 74.30%; hydrogen (H), 3.95%; N, 16.91%; S, 4.84%; test value: C, 74.30%; H, 3.94%; N, 16.93%; S, 4.83%. ESI-MS(m/z)(M⁺) is obtained through liquid chromatography-mass spectrometry: a theoretical value is 662.20 and a test value is 662.76.

Compounds H1, H2 and H5 are obtained with methods similar to the method for synthesizing the compound H3.

Synthesis of a compound H4

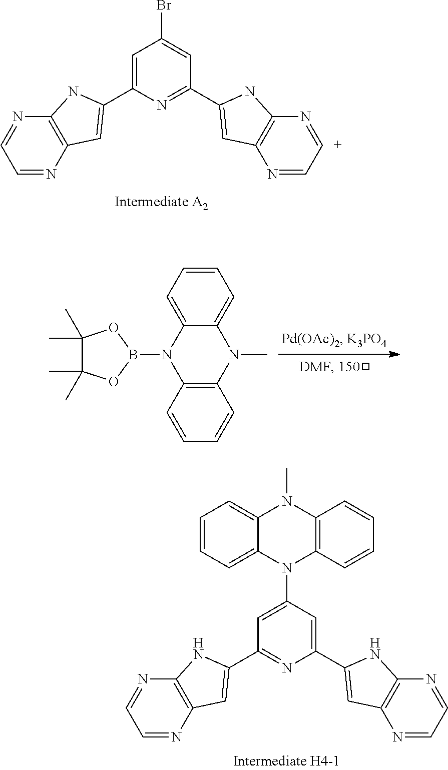

Step 1: nitrogen is fed into a 250 ml three-necked flask, an intermediate A₂ (0.03 mol), phenazine borate (0.036 mmol) and palladium acetate (0.0003 mol) are sequentially added into 150 ml of DMF, stirring for mixing is performed during the adding, then aqueous solution of K₃PO₄ (0.045 mol) is added, reflux reaction is performed for 10 h at a temperature of 130° C., natural cooling to room temperature is performed, 100 ml of water is added, an obtained mixture is filtered and dried in a vacuum drying oven, and obtained residues are enabled to pass through a silica gel column for further separation and purification to obtain an intermediate H4-1.

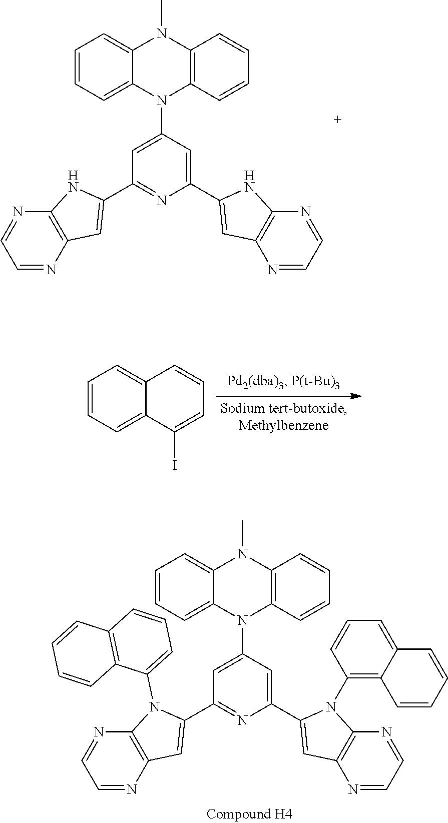 [SMILES: CN1c2ccccc2N(c2cc(-c3cc4nccnc4[nH]3)nc(-c3cc4nccnc4[nH]3)c2)c2ccccc21 ; Ic1cccc2ccccc12 ; CN1c2ccccc2N(c2cc(-c3cc4nccnc4n3-c3cccc4ccccc34)nc(-c3cc4nccnc4n3-c3cccc4ccccc34)c2)c2ccccc21]

Step 2: an intermediate H4-1 (0.03 mol), 4-bromo-pyridine (0.042 mol), Pd₂(dba)₃ (0.00018 mol), sodium tert-butoxide (0.00018 mol) and tri-tert-butylphosphine (0.00018 mol) are added into 100 ml of methylbenzene under the protection of nitrogen atmosphere, the mixture is stirred, and the mixed solution of the reactants is heated for refluxing for 10-15 h; sampling and plate spotting are performed till complete reaction; natural cooling and filtration are performed, rotary evaporation is performed to filtrate and the residue is enabled to pass through a silica gel column to obtain a compound H4.

Elementary analysis result of the compound H4 (molecular formula: C₅₀H₃₃N₉): theoretical value: C, 79.03%; H, 4.38%; N, 16.59%; test value: C, 79.03%; H, 4.36%; N, 16.61%. ESI-MS(m/z)(M⁺⁾ is obtained through liquid chromatography-mass spectrometry: a theoretical value is 759.29 and a test value is 759.86.

Synthesis of a compound H8

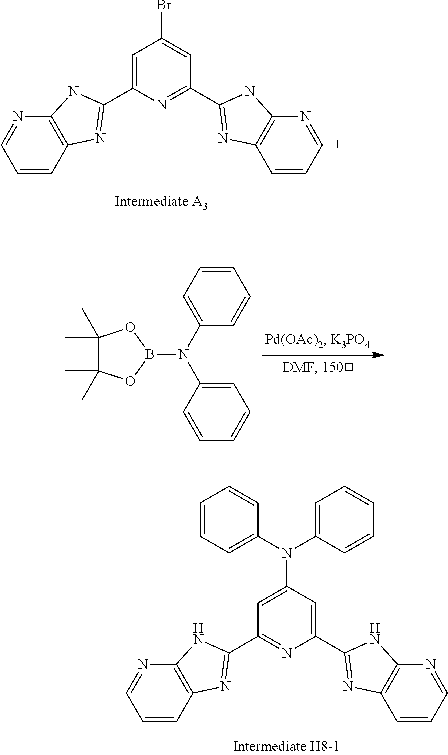

Step 1: nitrogen is fed into a 250 ml three-necked flask, an intermediate A₃ (0.03 mol), diphenylamine borate (0.036 mmol) and palladium acetate (0.0003 mol) are sequentially added into 150 ml of DMF, stirring for mixing is performed during the adding, then aqueous solution of K₃PO₄ (0.045 mol) is added, reflux reaction is performed for 10 h at a temperature of 130° C., natural cooling to room temperature is performed, 100 ml of water is added, an obtained mixture is filtered and dried in a vacuum drying oven, and obtained residues are enabled to pass through a silica gel column for further separation and purification to obtain an intermediate H8-1.

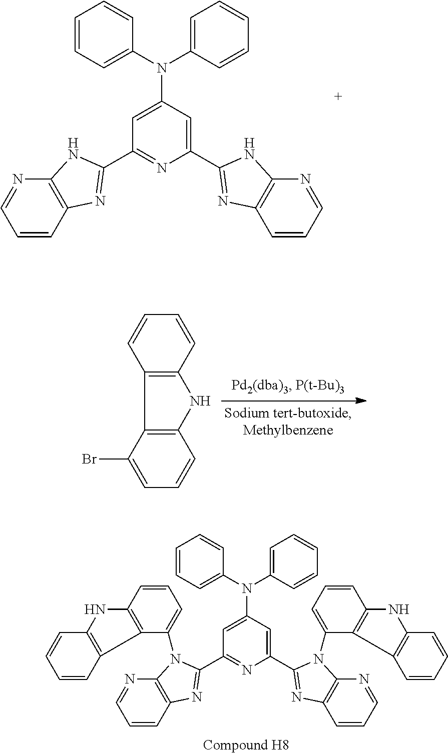 [SMILES: c1ccc(N(c2ccccc2)c2cc(-c3nc4cccnc4[nH]3)nc(-c3nc4cccnc4[nH]3)c2)cc1 ; Brc1cccc2[nH]c3ccccc3c12 ; c1ccc(N(c2ccccc2)c2cc(-c3nc4cccnc4n3-c3cccc4[nH]c5ccccc5c34)nc(-c3nc4cccnc4n3-c3cccc4[nH]c5ccccc5c34)c2)cc1]

Step 2: the intermediate H8-1 (0.03 mol), 4-bromo-pyridine (0.042 mol), Pd₂(dba)₃ (0.00018 mol), sodium tert-butoxide (0.00018 mol) and tri-tert-butylphosphine (0.00018 mol) are added into 100 ml of methylbenzene under the protection of nitrogen atmosphere, the mixture is stirred, and the mixed solution of the reactants is heated for refluxing for 10-15 h; sampling and plate spotting are performed till complete reaction; natural cooling and filtration are performed, rotary evaporation is performed to filtrate and the residue is enabled to pass through a silica gel column to obtain a compound H8.

Elementary analysis result of the compound H8 (molecular formula: C₅₃H₃₂N₁₀): theoretical value: C, 78.70%; H, 3.99%; N, 17.31%; test value: C, 78.70%; H, 4.01%; N, 17.29%. ESI-MS(m/z)(M⁺) is obtained through liquid chromatography-mass spectrometry: a theoretical value is 808.28 and a test value is 808.89.

Compounds H6, H7, H11 and H12 are obtained with methods similar to the method for synthesizing the compound H8.

Synthesis of a compound H9

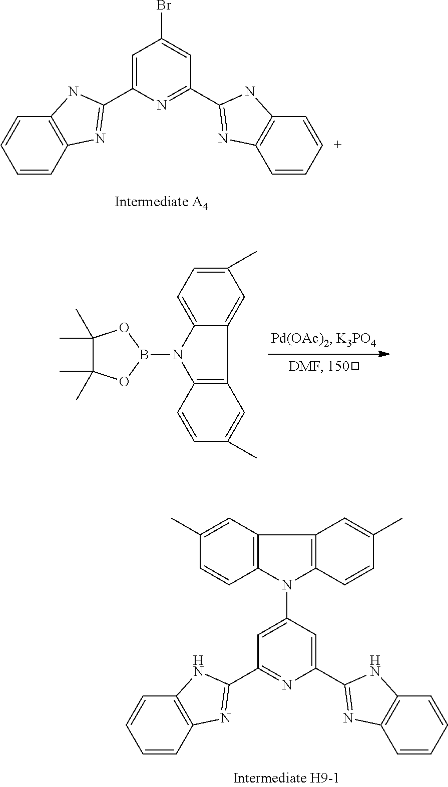

Step 1: nitrogen is fed into a 250 ml three-necked flask, an intermediate A₄ (0.03 mol), phenazine borate (0.036 mmol) and palladium acetate (0.0003 mol) are sequentially added into 150 ml of DMF, stirring for mixing is performed during the adding, then aqueous solution of K3PO4 (0.045 mol) is added, reflux reaction is performed for 10 h at a temperature of 130° C., natural cooling to room temperature is performed, 100 ml of water is added, an obtained mixture is filtered and dried in a vacuum drying oven, and obtained residues are enabled to pass through a silica gel column for further separation and purification to obtain an intermediate H9-1.

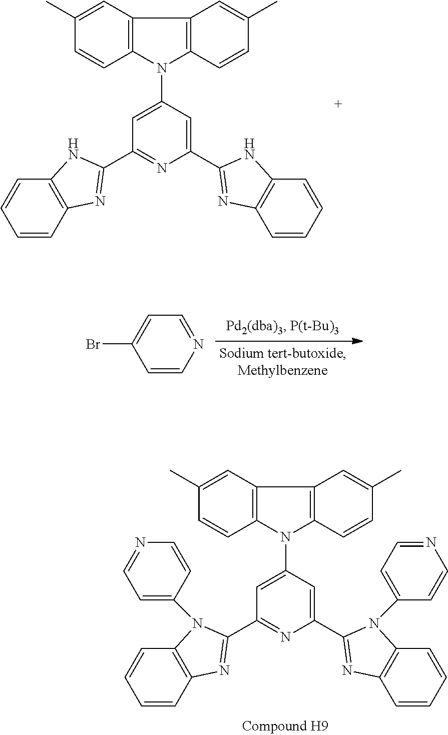 [SMILES: Cc1ccc2c(c1)c1cc(C)ccc1n2-c1cc(-c2nc3ccccc3[nH]2)nc(-c2nc3ccccc3[nH]2)c1 ; Brc1ccncc1 ; Cc1ccc2c(c1)c1cc(C)ccc1n2-c1cc(-c2nc3ccccc3n2-c2ccncc2)nc(-c2nc3ccccc3n2-c2ccncc2)c1]

Step 2: an intermediate H9-1 (0.03 mol), 4-bromo-pyridine (0.042 mol), Pd₂(dba)₃ (0.00018 mol), sodium tert-butoxide (0.00018 mol) and tri-tert-butylphosphine (0.00018 mol) are added into 100 ml of methylbenzene under the protection of nitrogen atmosphere, the mixture is stirred, and the mixed solution of the reactants is heated for refluxing for 10-15 h; sampling and plate spotting are performed till complete reaction; natural cooling and filtration are performed, rotary evaporation is performed to filtrate and the residue is enabled to pass through a silica gel column to obtain a compound H9.

Elementary analysis result of the compound H9 (molecular formula: C₄₃H₃₀N₈): theoretical value: C, 78.40%; H, 4.59%; N, 17.01%; test value: C, 78.40%; H, 4.57%; N, 17.03%. ESI-MS(m/z)(M⁺) is obtained through liquid chromatography-mass spectrometry: a theoretical value is 658.26 and a test value is 658.75.

A compound H10 is obtained with a method similar to the method for synthesizing the compound H9.

Synthesis of a compound H16

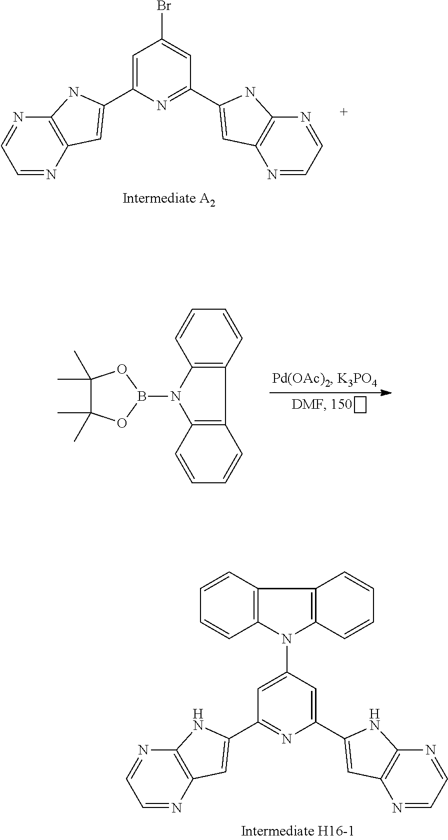 [SMILES: Brc1cc(-c2cc3nccnc3[nH]2)nc(-c2cc3nccnc3[nH]2)c1 ; CC1(C)OB(n2c3ccccc3c3ccccc32)OC1(C)C ; c1ccc2c(c1)c1ccccc1n2-c1cc(-c2cc3nccnc3[nH]2)nc(-c2cc3nccnc3[nH]2)c1]

Step 1: nitrogen is fed into a 250 ml three-necked flask, an intermediate A₂ (0.03 mol), carbazole borate (0.036 mmol) and palladium acetate (0.0003 mol) are sequentially added into 150 ml of DMF, stirring for mixing is performed during the adding, then aqueous solution of K₃PO₄ (0.045 mol) is added, reflux reaction is performed for 10 h at a temperature of 130° C., natural cooling to room temperature is performed, 100 ml of water is added, an obtained mixture is filtered and dried in a vacuum drying oven, and obtained residues are enabled to pass through a silica gel column for further separation and purification to obtain an intermediate H16-1.

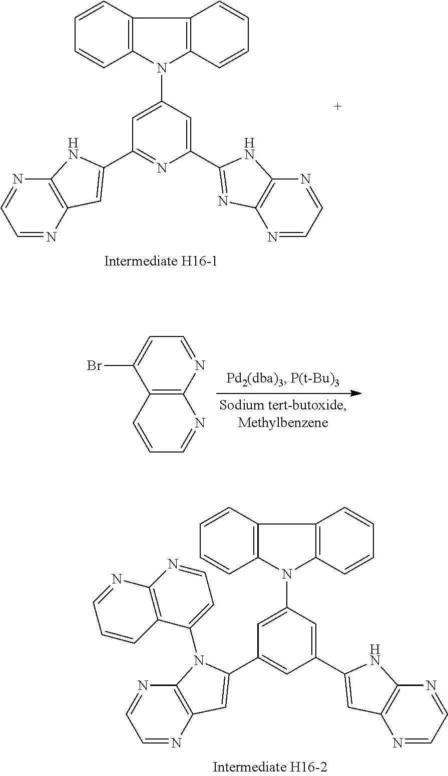 [SMILES: c1ccc2c(c1)c1ccccc1n2-c1cc(-c2cc3nccnc3[nH]2)nc(-c2nc3nccnc3[nH]2)c1 ; Brc1ccnc2ncccc12 ; c1cnc2nccc(-n3c(-c4cc(-c5cc6nccnc6[nH]5)cc(-n5c6ccccc6c6ccccc65)c4)cc4nccnc43)c2c1]

Step 2: an intermediate H16-1 (0.03 mol), 4-bromo-dipyridine (0.03 mol), Pd₂(dba)₃ (0.00018 mol), sodium tert-butoxide (0.00018 mol) and tri-tert-butylphosphine (0.00018 mol) are added into 100 ml of methylbenzene under the protection of nitrogen atmosphere, the mixture is stirred, and the mixed solution of the reactants is heated for refluxing for 10-15 h; sampling and plate spotting are performed till complete reaction; natural cooling and filtration are performed, rotary evaporation is performed to filtrate and the residue is enabled to pass through a silica gel column to obtain a compound H16-2.

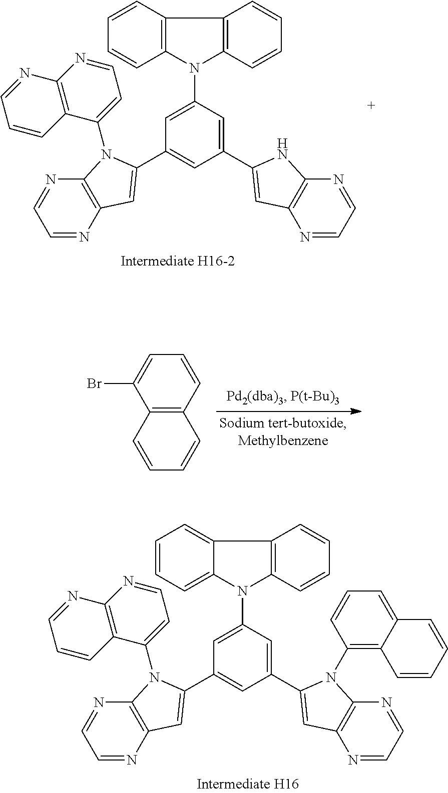 [SMILES: c1cnc2nccc(-n3c(-c4cc(-c5cc6nccnc6[nH]5)cc(-n5c6ccccc6c6ccccc65)c4)cc4nccnc43)c2c1 ; Brc1cccc2ccccc12 ; c1ccc2c(-n3c(-c4cc(-c5cc6nccnc6n5-c5ccnc6ncccc56)cc(-n5c6ccccc6c6ccccc65)c4)cc4nccnc43)cccc2c1]

Step 3: the intermediate H16-2 (0.03 mol), 4-bromo-naphthalene (0.03 mol), Pd₂(dba)₃ (0.00018 mol), sodium tert-butoxide (0.00018 mol) and tri-tert-butylphosphine (0.00018 mol) are added into 100 ml of methylbenzene under the protection of nitrogen atmosphere, the mixture is stirred, and the mixed solution of the reactants is heated for refluxing for 10-15 h; sampling and plate spotting are performed till complete reaction; natural cooling and filtration are performed, rotary evaporation is performed to filtrate and the residue is enabled to pass through a silica gel column to obtain a compound H16.

Elementary analysis result of the compound H16 (molecular formula: C₄₃H₃₀N₈): theoretical value: C, 78.78%; H, 3.99%; N, 17.23%; test value: C, 78.40%; H, 3.98%; N, 17.24%. ESI-MS(m/z)(M⁺) is obtained through liquid chromatography-mass spectrometry: a theoretical value is 731.8 and a test value is 731.75.

Compounds H13, H14 and H15 are obtained with methods similar to the method for synthesizing the compound H16.

In another aspect, the embodiment of the present disclosure provides an organic light emitting display device. The organic light emitting display device includes an organic electroluminescent device. The organic electroluminescent device includes an organic function layer. The organic function layer includes one or more organic film layers, at least one of the organic film layers is a light emitting layer, and the organic function layer contains the above-mentioned compound.

In one implementation, a phosphorescent host material of the light emitting layer contains the compound. When the light emitting material is used as a host material of the light emitting layer, the guest material is selected from BczVBi, coumarin-6, DCJTB, and etc.

In one implementation, a doping material of the light emitting layer contains the compound.

The organic function layer according to the embodiment of the present disclosure may further includes a hole injection layer (HIL), a hole transport layer (HTL), an electron blocking layer (EBL), a hole blocking layer (HBL), an electron transport layer (ETL) and an electron injection layer (EIL).

In one implementation, the organic function layer further includes an electron transport layer and a material of the electron transport layer contains the compound.

In one implementation, the organic function layer further includes a hole transport layer and a material of the hole transport layer contains the compound.

In one implementation, the organic function layer further includes a cap layer (CPL) and a material of the cap layer contains the compound.

In one implementation, the organic electroluminescent device further includes a first electrode and a second electrode over the organic function layer. The organic function layer is over the first electrode.

An anode material according to the embodiment of the present disclosure may be a metal such as copper, gold, silver, iron, chromium, nickel, manganese, palladium, platinum and etc., or may be an alloy such as metal oxide-indium oxide, zinc oxide, indium tin oxide (ITO), indium zinc oxide (IZO) and etc., or may be a conductive polymer such as polyaniline, polypyrrole, poly (3-methylthiophene) and etc. In addition to these materials and combinations thereof, which facilitate hole injection, the anode material may also be another material known to be suitable for manufacturing the anode.

A cathode material according to the embodiment of the present disclosure may be a metal such as aluminum, magnesium, silver, indium, tin, titanium and etc., or may be an alloy such as a multilayered metal material LiF/Al, LiO2/Al, BaF2/Al and etc. In addition to these materials and combinations thereof, which facilitate electron injection, the cathode material may also be another material known to be suitable for manufacturing the cathode.

The structure of the organic electroluminescent device according to the embodiment of the present disclosure is illustrated by FIG. 2. A substrate 1 is made of a glass or another suitable material (such as plastic); a first electrode 2 is a transparent electrode made of ITO, GZO and etc.; an organic function layer 3 includes one or more organic film layers; and a second electrode 4 is a metal cathode. The first electrode 2 and the second electrode 4 are interchangeable, i.e., the first electrode 2 can also be the metal cathode and correspondingly, the second electrode 4 can be the transparent electrode made of ITO, GZO and etc.

In an implementation, the organic electroluminescent device is prepared by adopting a vacuum evaporation method.

ITO/hole injection layer (thickness: 10 nm; material: molybdenum trioxide MoO₃)/hole transport layer (thickness: 80 nm; material: TAPC)/light emitting layer (thickness: 30 nm; material: compound H9 and Ir(ppy)₃, with a weight ratio of 100:10)/electron transport layer (thickness: 40 nm; material: TPBI compound)/electron injection layer (thickness: 1 nm; material: LiF)/Al.

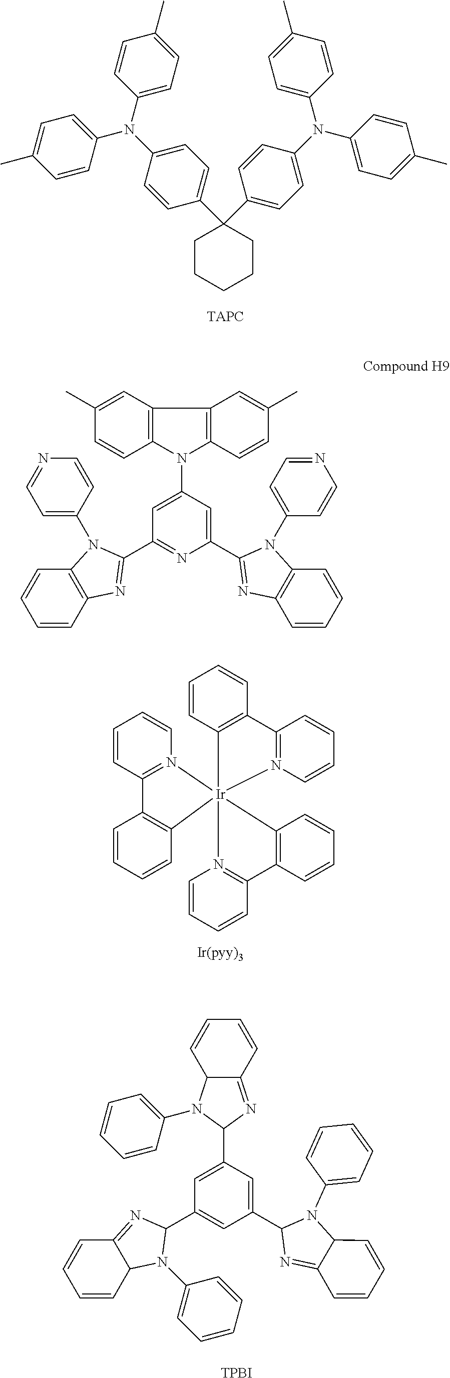

A specific preparation process is as follow. Washing is performed to an ITO transparent electrode (film thickness: 150 nm), i.e., alkali washing, pure water washing and drying are sequentially performed and then ultraviolet-ozone washing is performed to remove organic residues on the surface of the transparent ITO. Then, the washed ITO glass is mounted in a vacuum chamber, the reference pressure is regulated to 1*106 Torr, molybdenum trioxide MoO₃ having an evaporated film thickness of 10 nm is used as a hole injection layer, and then TAPC having a thickness of 80 nm is evaporated and used as a hole transport layer. After the evaporation of the hole transport material is ended, a light emitting layer of the OLED light emitting device is fabricated, the compound H9 according to the embodiment of the present disclosure is used as a host material, Ir(ppy)₃ is used as a doping material, a doping weight ratio is 1:10 and the total thickness of the light emitting layer is 300 nm. After the fabrication of the light emitting layer, a hole blocking/electron transport layer material TPBI having a thickness of 40 nm and an electron injection layer material lithium fluoride LiF having a film thickness of 1 nm are evaporated Finally, an aluminum (Al) layer having a film thickness of 80-100 nm is fabricated and used as a cathode reflecting electrode.

After the fabrication of the OLED light emitting device is completed, a known driving circuit is used for connecting the anode and the cathode, and the luminous efficiency, the luminescent spectrum and the current-voltage characteristics of the device are measured. The structural components of the obtained device are illustrated by FIG. 2; and the test results of the obtained device are shown by Table 3.

In one implementation, the organic electroluminescent device is prepared by adopting a solution processing method.

Specific steps for preparing a non-doped device includes: ultrasonic washing is performed to ITO glass twice by using acetone, alkaline washing liquor, ultrapure water and isopropanol, respectively, time for each washing is 15 min and then treatment is performed for 15 min by using an ozone washing machine; PEDOT: PSS solution is spin-coated onto the glass substrate to form a film layer of 40 nm, the glass substrate is placed in a vacuum drying oven for drying for 45 min at 120° C., then ortho-dichlorobenzene solution (concentration: 12 mg/mL) of the compound according to the embodiment of the present disclosure is coated and used as a light emitting layer, and the thickness is 40 nm; the substrate is transferred into a vacuum chamber for thermal evaporation coating to prepare an electron transport layer (TmPyPb, 50 nm), an electron injection layer (LiF, 0.5-1 nm) and a cathode (Al, 100 nm) to form a complete device.

Steps for preparing a doped device further include: ortho-dichlorobenzene solution (concentration: 12 mg/mL) of a host light emitting material and of a guest light emitting material is prepared separately, 50 μL (5%) of the guest material solution is moved and added into the host material solution by using a pipette, uniform magnetic stirring is performed and then the light emitting layer is coated. Other steps are the same as the specific steps for preparing the non-doped device.

In one implementation, the solution processing method is an inkjet printing method.

The organic light emitting display device according to embodiment of the present disclosure may be a display screen of a mobile phone, of a computer, of a liquid crystal television and etc. This is not specially restricted by this embodiment. FIG. 3 illustrates a schematic view of a mobile phone display screen, where 5 represents the display screen.

Accordingly, it can be seen that optional factors for the compound and the organic light emitting display device according to the embodiment of the present disclosure are many, and different embodiments may be obtained through combination according to the claims of the present disclosure. The embodiment of the present disclosure is only used for specific description of the present disclosure and is not used to restrict the present disclosure. The present disclosure will be further described below in combination with examples of organic electroluminescent devices containing the compound according to the embodiment of the present disclosure.

### EXAMPLES 1-16

The density functional theory (DFT) is applied for optimization and calculation to obtain distributions of molecular frontier orbits by using a Gaussian 09 program package under a B3LYP/6-31G(d) functional basis set aiming at the compound H1 to the compound H16.

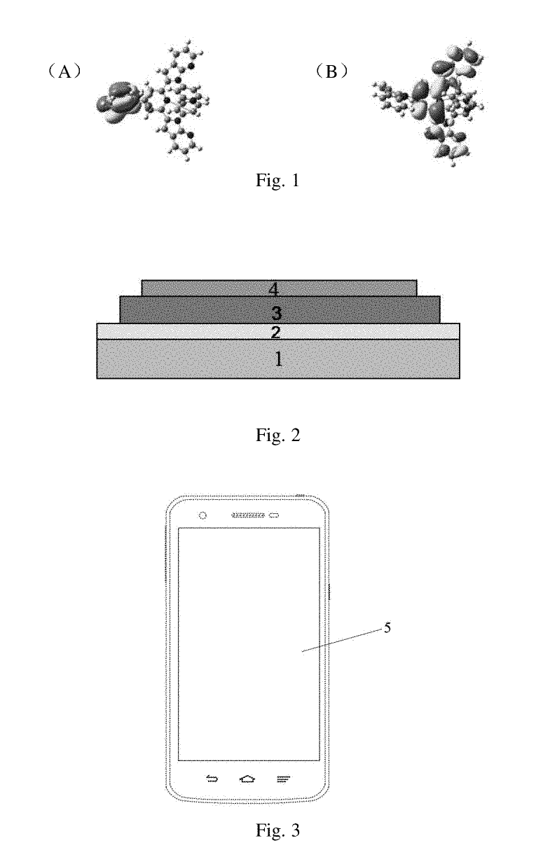

FIG. 1 illustrates orbit distributions of the compound H3, where (A) of FIG. 1 is an HOMO energy level distribution diagram of the compound H3 and (B) of FIG. 1 is an LUMO energy level distribution diagram of the compound H3. From FIG. 1, it can be seen that the HOMO and LUMO of the compound H3 are distributed in different units, respectively, better separating donors and receptors, thus facilitating the resonance between donor and receptor groups, decreasing the intersystem energy difference ΔE_{ST}, and improving the intersystem crossing ability from the singlet to the triplet.

Relevant data of examples 1-16 is as shown by Table 2. From Table 2, it can be seen that the triplet energy levels of the materials designed according to the embodiment of the present disclosure are high and are greater than 2.70 eV, and the ΔE_{ST} of all compounds are relatively small, thereby realizing relatively small singlet and triplet energy level differences, and facilitating carrier transport between organic function layers. (In Table 2, E_{T} represents the triplet energy level and ΔE_{ST} represents the difference between the singlet energy level and the triplet energy level.)

TABLE 2
Relevant performance data of compounds H1-H16

| | | HOMO | LUMO | E_{T} | ΔE_{ST} |
|---|---|---|---|---|---|
| Example | Compound | (ev) | (ev) | (ev) | (ev) |
| 1 | H1 | −5.75 | −3.14 | 2.73 | 0.16 |
| 2 | H2 | −5.61 | −3.20 | 2.82 | 0.32 |
| 3 | H3 | −5.84 | −3.02 | 2.79 | 0.64 |
| 4 | H4 | −5.63 | −2.98 | 2.88 | 0.44 |
| 5 | H5 | −5.80 | −3.14 | 2.76 | 0.25 |
| 6 | H6 | −5.74 | −3.18 | 2.73 | 0.35 |
| 7 | H7 | −5.69 | −2.85 | 2.81 | 0.09 |
| 8 | H8 | −5.83 | −3.06 | 2.69 | 0.37 |
| 9 | H9 | −5.67 | −3.27 | 2.74 | 0.48 |
| 10 | H10 | −5.82 | −3.06 | 2.73 | 0.26 |
| 11 | H11 | −5.69 | −3.09 | 2.80 | 0.13 |
| 12 | H12 | −5.88 | −3.04 | 2.82 | 0.38 |
| 13 | H13 | −−5.68 | −3.18 | 2.76 | 0.57 |
| 14 | H14 | −5.72 | −3.13 | 2.86 | 0.23 |
| 15 | H15 | −5.70 | −3.26 | 2.80 | 0.14 |
| 16 | H16 | −5.84 | −3.07 | 2.73 | 0.59 |

Devices Prepared by Adopting the Vacuum Evaporation Method

With the compound H1 as a light emitting material, a non-doped device N1 is designed and the structure of N1 is: [ITO(150 nm)]/[MoO₃(10 nm)]/[TAPC(80 nm)]/[H1(30 nm)]/[TPBI(40 nm)]/[LiF(1 nm)]/[Al(100 nm)].

With the compounds H1-H16 as host materials and Ir(ppy)₃ as a guest material, doped devices N1-N16 are designed and each of their structures is: [ITO(150 nm)]/[MoO₃(10 nm)]/[TAPC(80 nm)]/[H1(30 nm)]/[Ir(ppy)₃(50 nm)]/[TPBI(40 nm)]/[LiF(1 nm)]/[Al(100 nm)].

Relevant data of the above-mentioned devices is shown in Table 3. From Table 3, it can be seen that the non-doped device N1 using H1 as the host light emitting material achieves a maximum external quantum efficiency of 9.65%, this indicates that a symmetric ligation mode enables the material to present a highly twisted stereoscopic configuration, the steric hindrance is increased, the problem of triplet exciton annihilation caused by π-π stacking is greatly weakened and thus a more satisfactory device performance is obtained.

From Table 3, it can be seen that the maximum external quantum efficiency of the (doped) device N1 can reach 14.99%, which is about 45% higher than EQE₍ₘₐₓ₎ (%) of a non-doped device. This mainly benefits from the relatively high E_{T} of and the relatively small ΔE_{ST} characteristic of the material H1, transport and recombination of carriers between the host material and the guest material are facilitated and the device efficiency is improved. EQE₍ₘₐₓ₎ (%) of (doped) devices N1-N16 are all above 14% and the emitted blue light is pure. (In Table 3, V represents a working voltage, EL₍ₘₐₓ₎ represents a maximum current efficiency and EQE₍ₘₐₓ₎ represents a maximum external quantum efficiency, all from the evaporating equipment.)

TABLE 3
Results of performances of devices prepared by
adopting the vacuum evaporation method

| | Doped or | V | E_{L(max)} | EQE₍ₘₐₓ₎ |
|---|---|---|---|---|
| Device | non-doped | [V] | (cd A⁻¹) | (%) |
| N1 | Non-doped | 4.5 | 13.58 | 9.65 |
| N1 | Doped | 4.0 | 19.17 | 14.99 |
| N2 | Doped | 4.2 | 18.92 | 14.36 |
| N3 | Doped | 3.9 | 22.76 | 15.20 |
| N4 | Doped | 4.2 | 20.83 | 15.15 |
| N5 | Doped | 4.1 | 19.63 | 14.74 |
| N6 | Doped | 4.4 | 18.72 | 14.25 |
| N7 | Doped | 3.8 | 18.87 | 15.29 |
| N8 | Doped | 4.1 | 20.04 | 15.73 |
| N9 | Doped | 3.8 | 23.07 | 16.46 |
| N10 | Doped | 3.9 | 24.17 | 17.27 |
| N11 | Doped | 4.1 | 19.65 | 14.57 |
| N12 | Doped | 4.4 | 18.47 | 14.73 |
| N13 | Doped | 4.3 | 19.34 | 14.45 |
| N14 | Doped | 4.1 | 20.05 | 15.07 |
| N15 | Doped | 4.0 | 20.73 | 15.38 |
| N16 | Doped | 4.2 | 21.25 | 15.81 |

Devices Prepared by Adopting the Solution Method

With the compound H1 as a light emitting material, a non-doped device N17 is designed and the structure of N1 is: [ITO(100 nm)]/[PEDOT:PSS(40 nm)]/[H1(40 nm)]/[TmPyPb(50 nm)]/[LiF(0.5 nm)]/[Al(100 nm)].

With the compounds H1-H16 as host materials and BczVBi as a guest material, doped devices N17-N32 are designed and each of their structures is: [ITO(100 nm)]/[PEDOT:PSS(40 nm)]/[H1(40 nm)]/[BczVBi(50 nm)]/[TmPyPb(50 nm)]/[LiF(0.5 nm)]/[Al(100 nm)].

Relevant data of the above-mentioned devices is shown in Table 4. From Table 4, it can be seen that the non-doped device N17 using H1 as the host light emitting material achieves a maximum external quantum efficiency of 7.64%, this indicates that a proximity ligation mode enables the material to present a highly twisted stereoscopic configuration, the steric hindrance is increased, the problem of triplet exciton annihilation caused by π-π stacking is greatly weakened and thus a more satisfactory device performance is obtained.

From Table 4, it can be seen that the maximum external quantum efficiency of the (doped) device N17 can reach 12.02%, which is about 40% relative to EQE₍ₘₐₓ₎ (%) of a non-doped device. This mainly benefits from the relatively high E_{T} of and the relatively small ΔF_{ST} characteristic of the material H1, transport and recombination of carriers between the host material and the guest material are facilitated and the device efficiency is improved. EQE₍ₘₐₓ₎ (%) of (doped) devices N17-N32 are all above 11% and the emitted blue light is pure. (In Table 4, V represents a working voltage, EL₍ₘₐₓ₎ represents a maximum current efficiency and EQE₍ₘₐₓ₎ represents a maximum external quantum efficiency, all from the evaporating equipment.)

TABLE 4
Results of performance of devices prepared
by adopting the solution method

| | Doped or | V | E_{L(max)} | EQE₍ₘₐₓ₎ |
|---|---|---|---|---|
| Device | non-doped | [V] | (cd A⁻¹) | (%) |
| N17 | Non-doped | 4.6 | 11.15 | 7.64 |
| N17 | Doped | 4.1 | 16.82 | 12.02 |
| N18 | Doped | 4.3 | 16.18 | 12.54 |
| N19 | Doped | 4.1 | 17.67 | 12.87 |
| N20 | Doped | 4.3 | 17.69 | 13.10 |
| N21 | Doped | 4.3 | 16.13 | 12.06 |
| N22 | Doped | 4.2 | 15.96 | 11.54 |
| N23 | Doped | 4.0 | 18.01 | 12.78 |
| N24 | Doped | 4.2 | 15.26 | 11.93 |
| N25 | Doped | 3.9 | 19.15 | 12.76 |
| N26 | Doped | 4.0 | 21.03 | 13.35 |
| N27 | Doped | 4.0 | 16.48 | 11.74 |
| N28 | Doped | 4.2 | 15.07 | 11.92 |
| N29 | Doped | 4.5 | 14.54 | 11.88 |
| N30 | Doped | 4.4 | 14.09 | 11.34 |
| N31 | Doped | 4.2 | 15.82 | 12.03 |
| N32 | Doped | 4.4 | 15.46 | 12.61 |

The compound according to the embodiment of the present disclosure can be used as a host material of an organic electroluminescent device or a material of a CPL. Comparing with existing phosphorescent host materials, the compound according to the embodiment of the present disclosure has a higher triplet energy level E_{T}, a greater molecular density, a higher glass transition temperature and a better heat stability, which effectively improve the balanced transfer of carriers and widen the exciton recombination area, thus effectively improving the light extraction efficiency, lowering the driving voltage and greatly improving the luminous efficiency and lifetime of the device. Therefore, the compound can be well applied to the technical field of electroluminescent devices.

The compound according to the embodiment of the present disclosure can also be used as a doping material, a material of a hole transport layer or a material of an electron transport layer, in an organic electroluminescent device, which could reduce the driving voltage, and improve the luminous efficiency, brightness, heat stability, color purity and device lifetime.

Obviously, one skilled in the art may make various modifications and variations to the present disclosure without departing from the spirit and scope of the present disclosure. Therefore, if these modifications and variations to the present disclosure fall into the scope of the claims and their equivalents, the present disclosure is also intended to include these modifications and variations.

What is claimed is:

1. A compound, having a structure illustrated by [chemical formula 1]:
[chemical formula 1] is 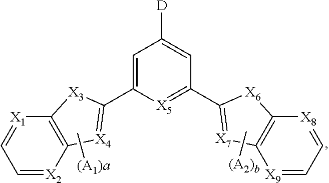
wherein D represents an electron donor unit, each of X₁-X₉ is independently selected from a carbon atom or a nitrogen atom, at least one of X₃ and X₄ is a nitrogen atom, at least one of X₆ and X₇ is a nitrogen atom, each of a and b in the subscripts is independently selected from 0 or 1, and each of A₁ and A₂ is independently selected from a group consisting of a substituted or an unsubstituted C₁-C₂₀ alkyl, a substituted or an unsubstituted C₃-C₂₀ cycloalkyl, a substituted or an unsubstituted C₁-C₂₀ alkoxy, a substituted or an unsubstituted C₃-C₂₀ heterocyclic, a substituted or an unsubstituted C₆-C₄₀ aryl, a substituted or an unsubstituted C₁₀-C₄₀ fused aromatic group, and a substituted or an unsubstituted C₄-C₄₀ heteroaryl.

2. The compound according to claim 1, wherein X₃ and X₆ are same groups, X₄ and X₇ are same groups, X₁ and X₈ are same groups and X₂ and X₉ are same groups.

3. The compound according to claim 1, wherein the compound is selected from one of following: 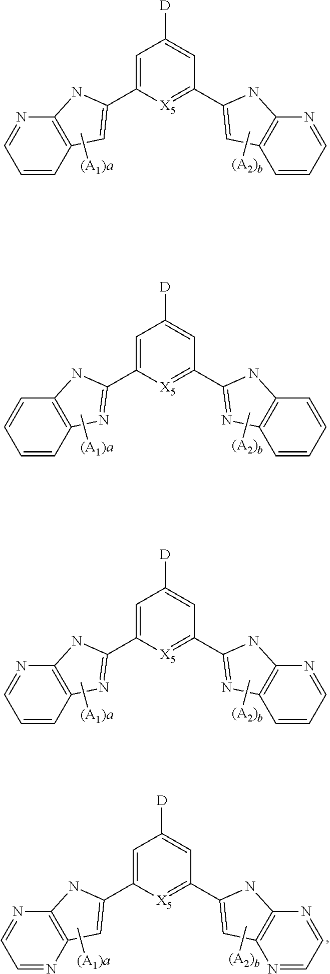
wherein D represents an electron donor unit, X₅ is selected from a carbon or a nitrogen atom, each of a and b in the subscripts is independently selected from 0 or 1, and each of A₁ and A₂ is independently selected from a group consisting of a substituted or an unsubstituted C₁-C₂₀ alkyl, a substituted or an unsubstituted C₃-C₂₀ cycloalkyl, a substituted or an unsubstituted C₁-C₂₀ alkoxy, a substituted or an unsubstituted C₃-C₂₀ heterocyclic, a substituted or an unsubstituted C₆-C₄₀ aryl, a substituted or an unsubstituted C₁₀-C₄₀ fused aromatic group, and a substituted or an unsubstituted C₄-C₄₀ heteroaryl.

4. The compound according to claim 1, wherein the unit D is selected from a group consisting of an anilino, an aniline derivative substituent, a carbazolyl, a carbazole derivative substituent, an acridinyl and an acridine derivative substituent.

5. The compound according to claim 1, wherein the unit D is selected from one of following: 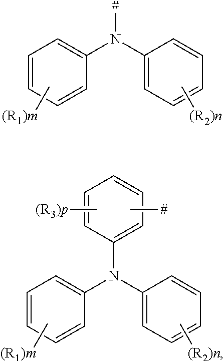
wherein each of m, n and p in the subscripts is independently selected from 0, 1, 2 or 3; the symbol # represents a location of connection with the heteroaryl in [chemical formula 1]; and each of R₁, R₂ and R₃ is independently selected from a group consisting of a hydrogen atom, a substituted or an unsubstituted C₁-C₃₀ alkyl, a substituted or an unsubstituted silylene, a substituted or an unsubstituted C₁-C₃₀ alkoxy, a substituted or an unsubstituted C₆-C₃₀ aryl, and, a substituted or an unsubstituted C₁₀-C₃₀ fused aromatic group.

6. The compound according to claim 1, wherein the unit D is selected from one of following: 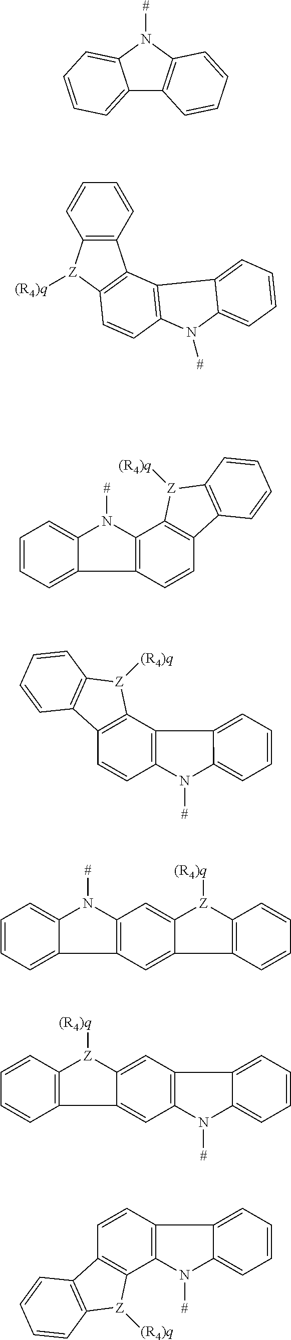
wherein Z is selected from a carbon atom, a nitrogen atom, an oxygen atom, a sulfur atom or a silicon atom; each of q in the subscripts is independently selected from 0, 1, 2 or 3; the symbol # represents a location linked with the heteroaryl in [chemical formula 1]; and R₄ is independently selected from a group consisting of a hydrogen atom, a substituted or an unsubstituted C₁-C₃₀ alkyl, a substituted or an unsubstituted silylene, a substituted or an unsubstituted C₁-C₃₀ alkoxy, a substituted or an unsubstituted C₆-C₃₀ aryl and a substituted or an unsubstituted C₁₀-C₃₀ fused aromatic group.

7. The compound according to claim 1, wherein the unit D is selected from one of following: 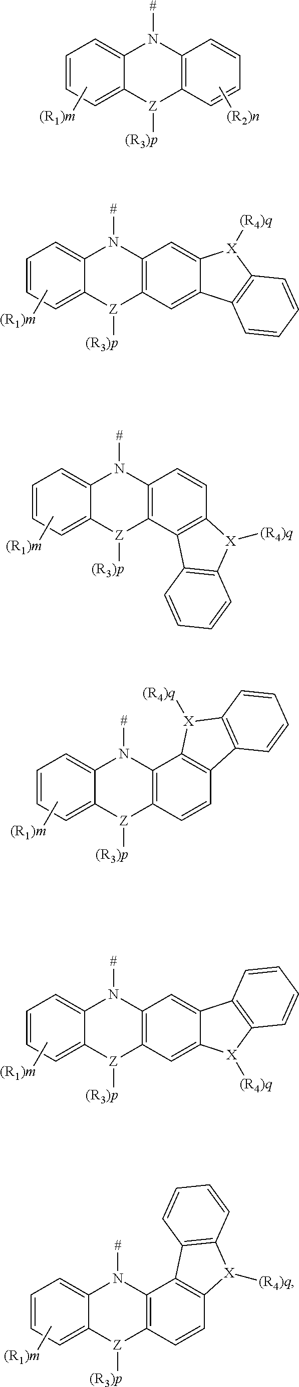
wherein Z is selected from a carbon atom, a nitrogen atom, an oxygen atom, a sulfur atom or a silicon atom; X is selected from a group consisting of a carbon atom, a nitrogen atom, an oxygen atom and a sulfur atom; each of m, n, p and q in the subscripts is independently selected from 0, 1, 2 or 3; the symbol # represents a location linked with the heteroaryl in [chemical formula 1]; and each of R₁, R₂, R₃ and R₄ is independently selected from a group consisting of a hydrogen atom, a substituted or an unsubstituted C₁-C₃₀ alkyl, a substituted or an unsubstituted silylene, a substituted or an unsubstituted C₁-C₃₀ alkoxy, a substituted or an unsubstituted C₆-C₃₀ aryl and a substituted or an unsubstituted C₁₀-C₃₀ fused aromatic group.

8. The compound according to claim 1, wherein the unit D is selected from one of following: 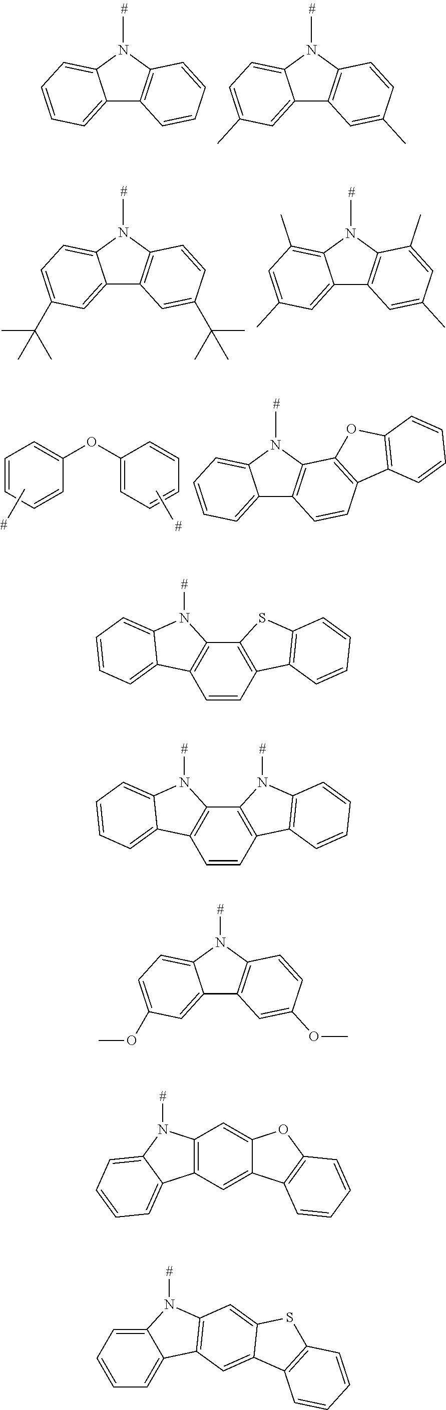 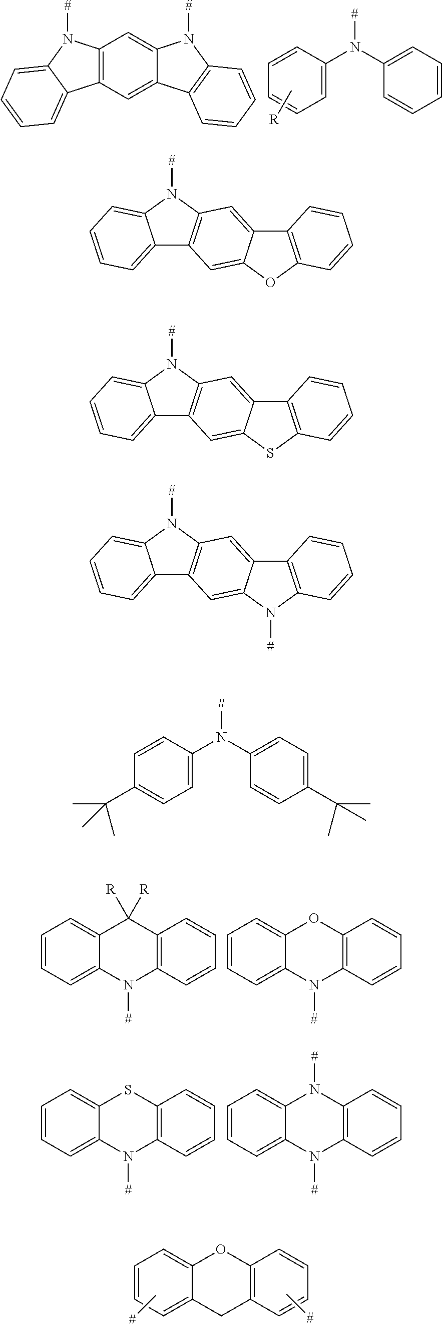 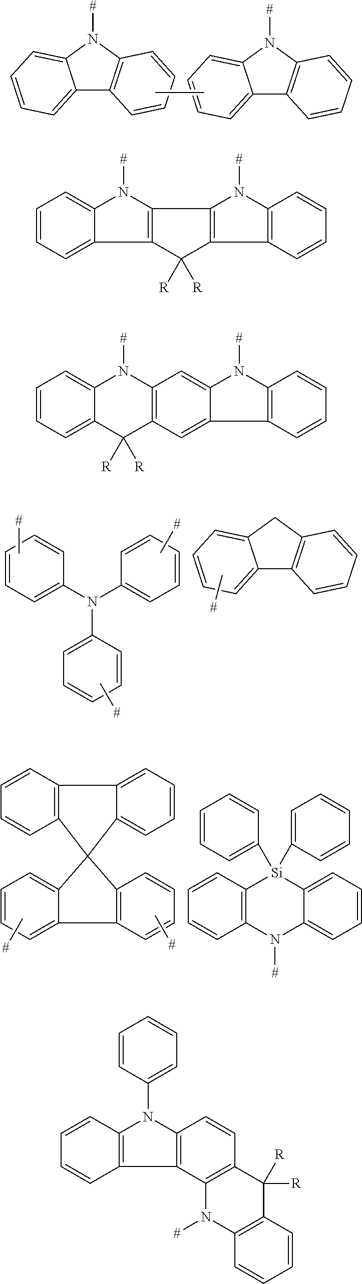 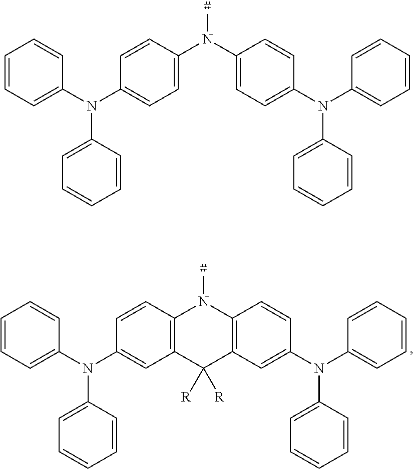
wherein R in each structural formula is independently selected from a group consisting of an hydrogen atom, a substituted or an unsubstituted C₁-C₂₀ alkyl, a substituted or an unsubstituted C₃-C₂₀ cycloalkyl, a substituted or an unsubstituted C₁-C₂₀ alkoxy, a substituted or an unsubstituted C₃-C₂₀ heterocyclic, a substituted or an unsubstituted C₆-C₄₀ aryl and a substituted or an unsubstituted C₅-C₄₀ heteroaryl, and the symbol # represents a location where unit D is able to connect with the heteroaryl in [chemical formula 1].

9. The compound according to claim 1, wherein the compound is selected from one of following chemical compounds:

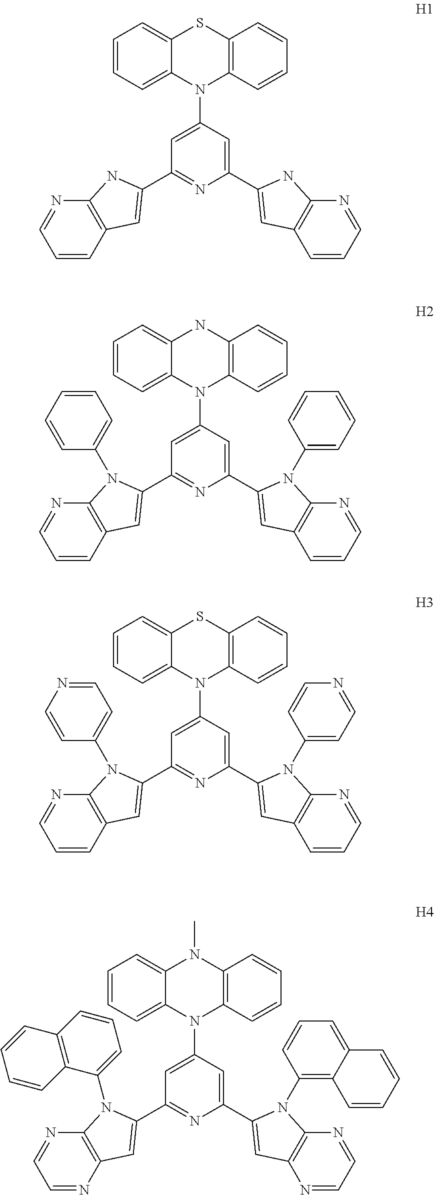 [SMILES: c1ccc2c(c1)Sc1ccccc1N2c1cc(-c2cc3cccnc3[nH]2)nc(-c2cc3cccnc3[nH]2)c1 ; c1ccc(-n2c(-c3cc(N4c5ccccc5Nc5ccccc54)cc(-c4cc5cccnc5n4-c4ccccc4)n3)cc3cccnc32)cc1 ; c1ccc2c(c1)Sc1ccccc1N2c1cc(-c2cc3cccnc3n2-c2ccncc2)nc(-c2cc3cccnc3n2-c2ccncc2)c1 ; CN1c2ccccc2N(c2cc(-c3cc4nccnc4n3-c3cccc4ccccc34)nc(-c3cc4nccnc4n3-c3cccc4ccccc34)c2)c2ccccc21]

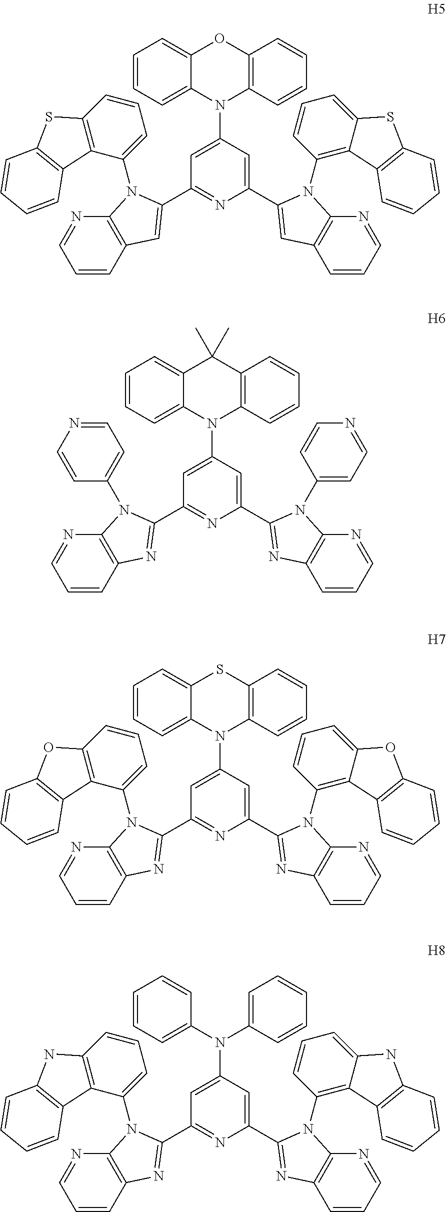 [SMILES: c1ccc2c(c1)Oc1ccccc1N2c1cc(-c2cc3cccnc3n2-c2cccc3sc4ccccc4c23)nc(-c2cc3cccnc3n2-c2cccc3sc4ccccc4c23)c1 ; CC1(C)c2ccccc2N(c2cc(-c3nc4cccnc4n3-c3ccncc3)nc(-c3nc4cccnc4n3-c3ccncc3)c2)c2ccccc21 ; c1ccc2c(c1)Sc1ccccc1N2c1cc(-c2nc3cccnc3n2-c2cccc3oc4ccccc4c23)nc(-c2nc3cccnc3n2-c2cccc3oc4ccccc4c23)c1 ; c1ccc(N(c2ccccc2)c2cc(-c3nc4cccnc4n3-c3cccc4[nH]c5ccccc5c34)nc(-c3nc4cccnc4n3-c3cccc4[nH]c5ccccc5c34)c2)cc1]

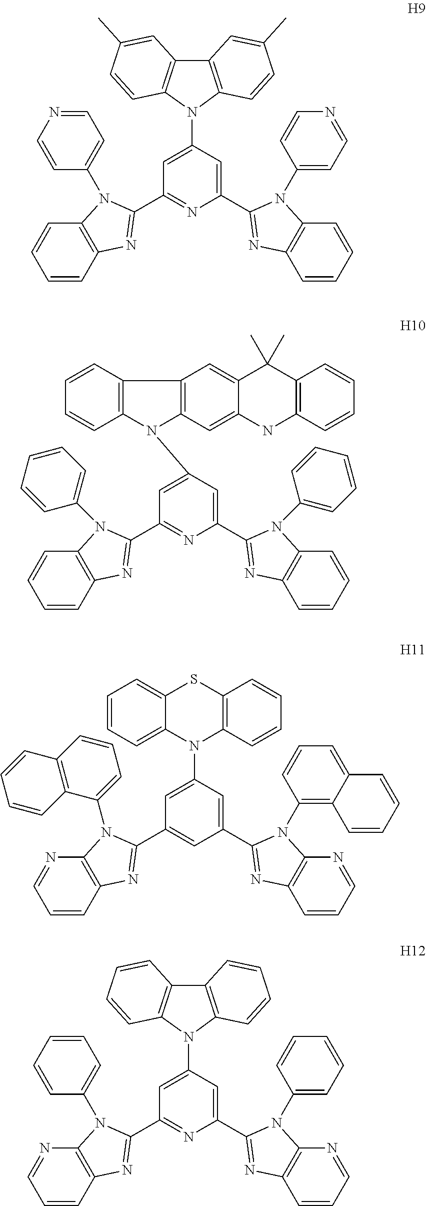 [SMILES: Cc1ccc2c(c1)c1cc(C)ccc1n2-c1cc(-c2nc3ccccc3n2-c2ccncc2)nc(-c2nc3ccccc3n2-c2ccncc2)c1 ; CC1(C)c2ccccc2Nc2cc3c(cc21)c1ccccc1n3-c1cc(-c2nc3ccccc3n2-c2ccccc2)nc(-c2nc3ccccc3n2-c2ccccc2)c1 ; c1ccc2c(c1)Sc1ccccc1N2c1cc(-c2nc3cccnc3n2-c2cccc3ccccc23)cc(-c2nc3cccnc3n2-c2cccc3ccccc23)c1 ; c1ccc(-n2c(-c3cc(-n4c5ccccc5c5ccccc54)cc(-c4nc5cccnc5n4-c4ccccc4)n3)nc3cccnc32)cc1]

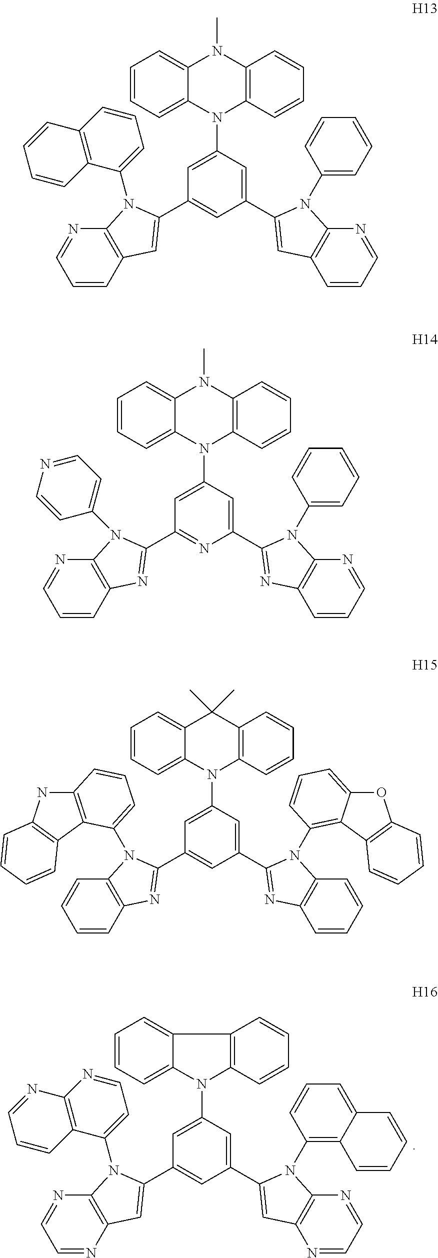 [SMILES: CN1c2ccccc2N(c2cc(-c3cc4cccnc4n3-c3ccccc3)cc(-c3cc4cccnc4n3-c3cccc4ccccc34)c2)c2ccccc21 ; CN1c2ccccc2N(c2cc(-c3nc4cccnc4n3-c3ccccc3)nc(-c3nc4cccnc4n3-c3ccncc3)c2)c2ccccc21 ; CC1(C)c2ccccc2N(c2cc(-c3nc4ccccc4n3-c3cccc4[nH]c5ccccc5c34)cc(-c3nc4ccccc4n3-c3cccc4oc5ccccc5c34)c2)c2ccccc21 ; c1ccc2c(-n3c(-c4cc(-c5cc6nccnc6n5-c5ccnc6ncccc56)cc(-n5c6ccccc6c6ccccc65)c4)cc4nccnc43)cccc2c1]

10. A method for preparing the compound according to claim 1, comprising the following steps expressed with [chemical reaction formula 1] to [chemical reaction formula 4]:
step 1, activating a 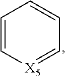
step 2, reacting with an activated 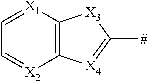
and an activated 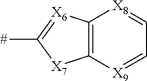
to obtain an intermediate I; wherein the intermediate I reacts with an activated group D to obtain an intermediate II; and wherein the intermediate II reacts with an activated A₁ and an activated A₂ to obtain the compound;
wherein
[chemical reaction formula 1] is 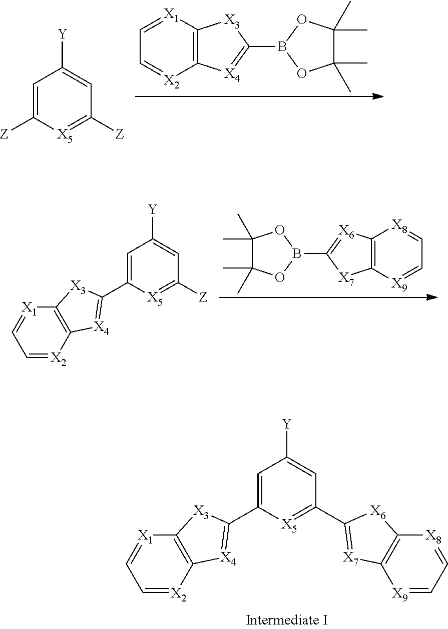
[chemical reaction formula 2] is 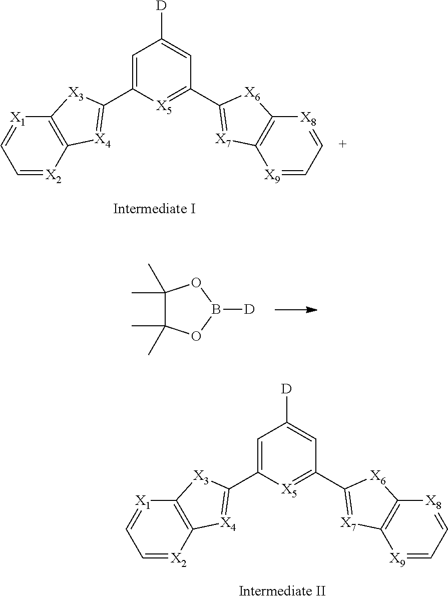
[chemical reaction formula 3] is 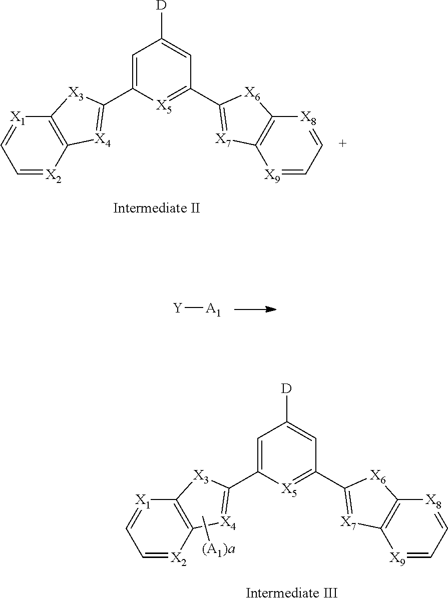
[chemical reaction formula 4] is 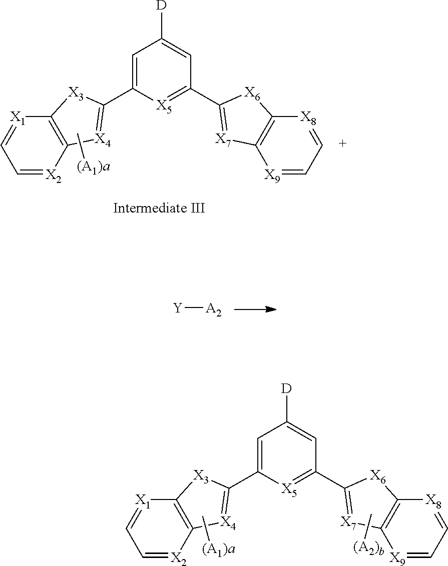
wherein Y and Z represent two different halogens, respectively, Y is selected from iodine or bromine, and Z is selected from bromine or chlorine.

11. An organic light emitting display device, comprising an organic electroluminescent device,
wherein the organic electroluminescent device comprises an organic function layer;
wherein the organic function layer comprises one or more organic film layers;
wherein at least one of the organic film layers is a light emitting layer; and
wherein the organic function layer contains the compound according to claim 1.

12. The organic light emitting display device according to claim 11, wherein a phosphorescent host material of the light emitting layer contains the compound.

13. The organic light emitting display device according to claim 11, wherein a doping material of the light emitting layer contains the compound.

14. The organic light emitting display device according to claim 11, wherein the organic function layer further comprises an electron transport layer and a material of the electron transport layer contains the compound.

15. The organic light emitting display device according to claim 11, wherein the organic function layer further comprises a hole transport layer and a material of the hole transport layer contains the compound.

16. The organic light emitting display device according to claim 11, wherein the organic function layer further comprises a cap layer and a material of the cap layer contains the compound.

17. The organic light emitting display device according to claim 11, wherein the organic electroluminescent device further comprises a first electrode and a second electrode over the organic function layer, and the organic function layer is over the first electrode.
